(19) United States
(12) Patent Application Publication
(10) Pub. No.: US 2025/0201338 A1
(43) Pub. Date: Jun. 19, 2025

(54) METHODS FOR CLASSIFYING, DETECTING AND TREATING BIOLOGICAL DISEASES

(71) Applicant: FBB BIOMED, INC., Coralville, IA (US)

(72) Inventors: Howard B. URNOVITZ, Coralville, IA (US); Lindsey E. KASCHMITTER, Coralville, IA (US); Julien F. S. REY, Coralville, IA (US)

(73) Assignee: FBB BIOMED, INC., Coralville, IA (US)

(21) Appl. No.: 18/980,579

(22) Filed: Dec. 13, 2024

Related U.S. Application Data
(60) Provisional application No. 63/701,929, filed on Oct. 1, 2024.
(60) Provisional application No. 63/701,962, filed on Oct. 1, 2024.
(60) Provisional application No. 63/609,719, filed on Dec. 13, 2023.
(60) Provisional application No. 63/609,756, filed on Dec. 13, 2023.
(60) Provisional application No. 63/730,857, filed on Dec. 11, 2024.

(51) Int. Cl. G16B 20/00; C12Q 1/6883; G16B 25/10; G16B 40/20; G16H 50/20

(52) U.S. Cl. CPC G16B 20/00; C12Q 1/6883; G16B 25/10; G16B 40/20; G16H 50/20; C12Q 2600/158

(57) ABSTRACT

The current disclosure provides for methods and compositions for classifying subjects having different biological states. The disclosure describes a method comprising: filtering sequence data obtained from a sample from a subject based on long non-coding RNA (lncRNA) and/or pseudogene RNA (pgRNA), and/or the reference genome; determining a biological state classification of the subject by providing the filtered sequence data to one or more machine learning classifiers as input, wherein the one or more machine learning classifiers is trained to output biological state classifications based on filtered sequence data of a training data set.

### CROSS REFERENCE TO RELATED APPLICATIONS

This application claims the benefit of priority of U.S. Provisional Patent Application Ser. No. 63/609,719 filed Dec. 13, 2023, U.S. Provisional Patent Application Ser. No. 63/609,756 filed Dec. 13, 2023, U.S. Provisional Patent Application Ser. No. 63/701,929 filed Oct. 1, 2024, U.S. Provisional Patent Application Ser. No. 63/701,962 filed Oct. 1, 2024, and U.S. Provisional Patent Application Ser. No. 63/730,857 filed Dec. 11, 2024, the contents of each of which are hereby incorporated by reference in their entirety.

### TECHNICAL FIELD AND BACKGROUND

### I. Field of the Invention

The present invention relates generally to the fields of classification of two or more different states based on a machine learning model applied to analyze sequence data from biological samples from a subject.

### II. Background

Making a medical diagnosis involves identifying the disease or conditions that explain a person's symptoms and signs. Typically, diagnostic information is gathered from the patient's history and physical examination. It is frequently difficult due to the fact that many indications and symptoms are ambiguous and can be diagnosed only by trained health experts. Moreover, as humans are prone to error or that there is not enough information, it is not surprising that a patient may have overdiagnosis or misdiagnosis occur more often. The use of machine learning (ML) has gained popularity in the medical field, including disease diagnosis in health care. While traditional diagnosis processes can be costly, time-consuming, and often require human intervention, ML-based methods have the possibility of providing more inexpensive and time-effective means for classifying different states, such as different disease states. Thus, there is a need in the art for methods more accurately and/or more quickly assess a patient, including methods that incorporate machine learning for classifying subjects of different states.

### SUMMARY

The current disclosure provides for methods and compositions for classifying subjects having different biological states. As described herein, endogenous ancestral nucleic sequences (EANS) can be used to determine biological states in humans and nonhumans. There is an abundance of ignored genetic elements as diagnostic targets. In fact, often these elements are discarded when samples of nucleic acids are evaluated. They encompass a vast array of categories, including endogenous viruses, retrotransposons, transfer RNA (tRNA), ribosomal RNA (rRNA), microRNAs (miRNAs), long non-coding RNA (lncRNAs), pseudogenes, piwi-interacting RNA (piRNAs), and ribonuclease P RNA. The inventors have discovered that enriching RNA samples for eansRNA can be a meaningful step in a process that can be utilized to diagnose and prognose diseases in mammalian subjects. In combination with machine learning, it is even more powerful. These tools can also be used in many other aspects, such as for monitoring disease progression, predicting disease severity, and/or for determining treatment efficacy.

The disclosure describes a method comprising predicting, determining, or analyzing a biological state classification of a subject by inputting sequence data obtained from a sample from a subject to one or more machine learning classifiers as input, wherein the one or more machine learning classifiers is trained to predict or output biological state classifications based on sequence data of a training data set; wherein the sequence data comprises the sequences of the RNA isolated from a sample that has been depleted of linear RNA or enriched for double stranded RNA. Also described is a method comprising: filtering sequence data obtained from a sample from a subject based on long non-coding RNA (lncRNA) and/or pseudogene RNA (pgRNA), and/or the reference genome; predicting, determining, or analyzing a biological state classification of the subject by inputting the filtered sequence data to one or more machine learning classifiers as input, wherein the one or more machine learning classifiers is trained to predict or output biological state classifications based on filtered sequence data of a training data set. The methods may comprise or further comprise outputting the biological state classification of the subject.

Also described is a method for treating a subject for a disease state, the method comprising treating a subject for the disease state, wherein the subject has been determined to have the disease state by a trained machine learning classifier that distinguishes between subjects having different disease states and/or subjects not having a disease state, wherein the machine learning classifier is trained on sequence data filtered by lncRNA, and/or pgRNA, and/or the reference genome and provided from a sample from subjects having a first disease state and of subjects not having a first disease state. The disclosure also provides for a method comprising: i) sequencing a biological sample from a subject comprising isolated RNA; and/or ii) detecting the amount of RNA fragments in the sample that comprises RNA sequences having at least 95% sequence identity to lncRNA, and/or pgRNA, and/or the reference genome.

Also described is a method for monitoring, diagnosing, and/or prognosing a subject with a disease, the method comprising: detecting the amount of RNA fragments in a sample from the subject that comprise RNA sequences having at least 95% sequence identity to lncRNA, and/or pgRNA, and/or the reference genome. Also provided is a method for monitoring, diagnosing, and/or prognosing a subject with a disease, the method comprising: detecting the amount of RNA fragments in a sample from the subject that comprise RNA sequences having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity (or any derivable range therein) to lncRNA, and/or pgRNA, and/or the reference genome. Also described is a method for monitoring, diagnosing, and/or prognosing a subject with a disease or for monitoring treatment efficacy and/or disease progression, monitoring, diagnosing, and/or prognosing the subject as having a certain disease state, wherein the subject has been determined to have the disease state by a trained machine learning classifier that is trained to predict or output biological state classifications based on sequence data of a training data set; wherein the sequence data comprises the sequences of the RNA isolated from a sample that has been depleted of linear RNA or enriched for double stranded RNA

Also provided is a method for treating a subject for a disease state, the method comprising treating a subject determined to have an increase or decrease in disease-associated RNA fragments in a sample from the subject compared to a control, wherein the disease-associated RNA fragments comprise RNA fragments that have at least 95% sequence identity to lncRNA, and/or pgRNA, and/or the reference genome. Also described is a method for treating a subject for a disease state, the method comprising treating a subject determined to have an increase or decrease in disease-associated RNA fragments in a sample from the subject compared to a control, wherein the disease-associated RNA fragments comprise RNA fragments that have, have at least, or have at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity (or any derivable range therein) to lncRNA, and/or pgRNA, and/or the reference genome. Also provided is a method for treating a subject for a disease state, the method comprising treating a subject for the disease state, wherein the subject has been determined to have the disease state by a trained machine learning classifier that is trained to predict or output biological state classifications based on sequence data of a training data set; wherein the sequence data comprises the sequences of the RNA isolated from a sample that has been depleted of linear RNA or enriched for double stranded RNA.

Also described is a kit comprising one or more reagents for stabilizing RNA and an exo-ribonuclease that preferentially hydrolyzes single-stranded RNA.

Methods include inputting nucleic acid sequence data obtained or determined from a biological sample from a subject to one or more machine learning classifiers, wherein the one or more machine learning classifiers is trained to output whether a subject has a neurological disease based on sequence data of a training data set; and outputting a neurological disease state of the subject, wherein the sequence data comprises the sequences of RNA isolated from the subject's sample that has been depleted of linear RNA through digestion of linear RNA that is followed by removal of the digested RNA; and wherein the neurological disease is Alzheimer's disease, multiple sclerosis, Parkinson's disease, mild cognitive impairment, multiple system atrophy, depression, diabetes neuropathy, dementia, schizophrenia, progressive supranuclear palsy, neurologic Long Covid, or amyotrophic lateral sclerosis. The methods may comprise (i) depleting linear RNA in a biological sample from a subject; wherein depleting linear RNA comprises contacting the biological sample with an exonuclease to digest linear RNA, followed by removal of the digested RNA; (ii) sequencing the remaining RNA in the sample depleted of linear RNA to generate sequence data; (iii) inputting the sequence data obtained from (ii) to one or more machine learning classifiers, wherein the one or more machine learning classifiers is trained to assess whether a subject has a neurological disease based on sequence data of a training data set; and outputting a neurological disease state of the subject; wherein the neurological disease is Alzheimer's disease, multiple sclerosis, Parkinson's disease, mild cognitive impairment, multiple system atrophy, depression, diabetes neuropathy, dementia, schizophrenia, progressive supranuclear palsy, neurologic Long Covid, or amyotrophic lateral sclerosis. Methods also include: i) depleting linear RNA in a biological sample from a subject; wherein depleting RNA comprises contacting the biological sample with an exonuclease to digest linear RNA, followed by removal of the digested RNA; ii) sequencing the RNA in the sample that has been depleted of linear RNA to generate sequence data; and iii) detecting the amount of RNA fragments in the sample that have RNA sequences with at least 95% sequence identity to lncRNA and/or pgRNA; wherein the sample that is depleted of linear RNA is a biological sample from one subject; wherein the RNA sample that is sequenced comprises 2-5 μg of RNA and less than 14% exonic RNA.

Also described is a method comprising: receiving a training data set comprising sequence data for each of a plurality of subjects having a known biological state classification, wherein the known biological state classification is one of having a first biological state or not having the first biological state; and training, using the training data set, one or more machine learning classifiers to output a biological state classification based on an inputted sequence data. The sequence data for each of the plurality of subjects having the known biological state classifications may comprise values for a set of parameters, wherein the training may comprise: reducing a dimensionality of the machine learning classifier based on a covariance of two or more parameters of the set of parameters.

The method may comprise or further comprise filtering sequence data obtained from a sample from a subject based on long non-coding RNA (lncRNA) and/or pseudogene RNA (pgRNA) and/or a reference genome. The method may comprise, further comprise, or exclude depleting linear RNA from the biological sample prior to sequencing. The method may further comprise training the machine learning classifier using training data, wherein the training data comprises a filtered sequence profile for a plurality of subjects having a first biological state and a plurality of subjects not having a second biological state. The machine learning classifier may comprise a machine learning classifier trained with training data, wherein the training data comprises a filtered sequence profile for a plurality of subjects having a first biological state and a plurality of subjects having a second biological state. The machine learning classifier may comprise a machine learning classifier trained with training data, wherein the training data comprises a filtered sequence profile for a plurality of subjects having a first biological state, a plurality of subjects having a second biological state, a plurality of subjects having a third biological state, a plurality of subjects having a fourth biological state, and a plurality of subjects having a fifth biological state. The machine learning classifier may comprise a machine learning classifier trained with training data, wherein the training data comprises a filtered sequence profile for a plurality of subjects having a first to nth biological state, wherein n is an integer between 1 and 100. n may be, be at least, or be at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100, or any derivable range therein. Predicting, determining, or analyzing a biological state classification may comprise: generating a report that identifies that the sample evidences the biological state classification.

The sample depleted of linear RNA may comprise or exclude double-stranded RNAs, circRNAs, rRNAs, tRNAs, miRNAs, snRNAs, and/or hairpin RNA. The sequence data or the RNA that is sequenced may comprise a GC content of greater than 55%. The sequence data or the RNA that is sequenced may comprise a GC content of, of less than, or of greater than 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or any derivable range therein. The sequence data or the RNA that is sequenced may comprise less than 14% exonic RNA. The sequence data or the RNA that is sequenced may comprise, comprise less than, or comprise more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60% exonic RNA, or any derivable range therein. The sequence data or the RNA that is sequenced may comprise, comprise less than, or comprise more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60% depleted linear RNA, or any derivable range therein.

The sequence data or the RNA that is sequenced may comprise the nucleotide sequences of RNA fragments that are 35-150 nucleotides in length. The sequence data or the RNA that is sequenced may comprise the nucleotide sequences of RNA fragments that are 35-500 nucleotides in length. The sequence data or the RNA that is sequenced may comprise the nucleotide sequences of RNA fragments that are, are at least, or are at most 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 nucleotides in length, or any derivable range therein.

The sequence data or the RNA that is sequenced may be from RNA extracted from about 1.5-4 mL of blood. The sequence data or the RNA that is sequenced may be from RNA extracted from about 0.5-10 mL of blood. The sequence data or the RNA that is sequenced may be from RNA extracted from about 1-3 mL of blood. The sequence data or the RNA that is sequenced may be from RNA extracted from, from at least, or from at most 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, or 15 mL of blood, or any derivable range therein. The sequence data or the RNA that is sequenced may exclude sequences from 3′ polyadenylated RNA. The sequence data or the RNA that is sequenced may exclude sequence from mechanical size-selected RNA. The methods may exclude 3′ polyadenylation. The methods may exclude mechanical size-selection of RNA and/or DNA. Mechanical size-selection can include size selection of nucleic acids by mechanical means, such as gel electrophoresis or bead-based size selection. The sequence data may comprise sequences from a sample that has been depleted of linear RNA. The reference genome may comprise a species-specific reference genome. The species of the reference genome assembly may be the same of the subject. The species of the reference genome assembly may be a species that is different than the subject. The species may comprise H. sapiens.

The methods may comprise sequencing 2-5 μg of RNA. The methods may comprise sequencing 1-10 μg of RNA. The methods may comprise sequencing 0.5-100 μg of RNA. The methods may comprise sequencing, sequencing at least, or sequencing at most 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μg of RNA, or any derivable range therein. The sequence data may be from, from at least, or from at most 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μg of sequenced RNA, or any derivable range therein.

The sample may be one that has been depleted of linear RNA by incubation of the RNA isolated from the sample with an exoribonuclease that preferentially hydrolyzes single-stranded RNA. The exoribonuclease may be one that hydrolyzes RNA in the 3′-5′ direction. The exoribonuclease may comprise RNAse R. RNase R, or Ribonuclease R, is a three to five prime exoribonuclease, which belongs to the RNase II superfamily, a group of enzymes that hydrolyze RNA in the 3′-5′ direction. RNase R has homologues in many other organisms. The amount of RNAse R used may be about 1-10 units per 2-20 μg RNA. The amount of RNAse R used may be, be at least, or be at most 0.2, 0.4, 0.6, 0.8, 1, 1.2, 1.4, 1.6, 1.8, 2, 2.2, 2.4, 2.6, 2.8, 3, 3.2, 3.4, 3.6, 3.8, 4, 4.2, 4.4, 4.6, 4.8, 5, 5.2, 5.4, 5.6, 5.8, 6, 6.2, 6.4, 6.6, 6.8, 7, 7.2, 7.4, 7.6, 7.8, 8, 8.2, 8.4, 8.6, 8.8, 9, 9.2, 9.4, 9.6, 9.8, 10, 10.2, 10.4, 10.6, 10.8, 11, 11.2, 11.4, 11.6, 11.8, 12, 12.2, 12.4, 12.6, 12.8, 13, 13.2, 13.4, 13.6, 13.8, 14, 14.2, 14.4, 14.6, 14.8, 15, 15.2, 15.4, 15.6, 15.8, 16, 16.2, 16.4, 16.6, 16.8, 17, 17.2, 17.4, 17.6, 17.8, 18, 18.2, 18.4, 18.6, 18.8, 19, 19.2, 19.4, 19.6, 19.8, or 20 Units (or any derivable rage therein) per, per at least, or per at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μg RNA (or any derivable range therein). The RNAse R may be isolated from an organism or synthetically made. The RNAse R may be from any source or may be an enzyme from a source that has been modified to enhance its activity. The RNAse R source may include or exclude Escherichia coli, Staphylococcus aureus, Salmonella enterica, Klebsiella pneumoniae, Acinetobacter baumannii, Mycobacterium tuberculosis, Pseudomonas aeruginosa, unclassified Streptomyces, Vibrio crassostreae, Streptococcus suis, Streptococcus pneumoniae, Xanthomonas oryzae, Enterobacter hormaechei, Streptomyces, Streptococcus agalactiae, Streptococcus pyogenes, Bacillus subtilis, Lactococcus lactis, Vibrio cholerae, and Vibrio parahaemolyticus. The sample may be one that is deplete of linear RNA. The sample may be depleted of at least 75% of linear RNA or of mRNA or of RNAse R substrates. The sample may be depleted of, depleted of at least, or depleted of at most 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% (or any derivable range therein) of linear RNA or of mRNA or of RNAse R substrates.

Depleting linear RNA may comprise incubating the RNA isolated from the sample with an exoribonuclease that preferentially hydrolyzes single-stranded RNA. Detecting the amount of RNA fragments in the sample may comprise counting the number of RNAs having at least 95% sequence identity to lncRNA and/or pgRNA and/or a reference genome.

Other methods for depleting linear RNA are known in the art and may be used in the methods of the disclosure. The depletion of linear RNA may include or exclude a nucleic acid-based capture method such as one that uses probes and/or beads, one that uses adaptors attached to a detection molecule, methods utilizing nanowell-based technologies, methods that utilize sieve-like or tunnel-like technologies (see, for example, Oxford nanopore technologies described in Zhao et al., Front. Microbiol. 14:1179966, which is incorporated by reference for all purposes), PCR-based methods, such as quantitative PCR and reverse transcription PCR, methods that include loop-mediated isothermal amplification, and methods utilizing microarrays. Depleting linear RNA may comprise or exclude selectively amplifying double-stranded RNA in order to reduce relative linear RNA presence.

The sequence data may be filtered to provide sequences having RNAs having at least 95% sequence identity to lncRNA, and/or pgRNA, and/or the reference genome. Detecting the amount of RNA fragments in the sample may comprise counting the number of RNAs having at least 95% sequence identity to lncRNA, and/or pgRNA, and/or the reference genome. The sequence data may be filtered to provide sequences having RNAs having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to lncRNA, and/or pgRNA, and/or the reference genome. Detecting the amount of RNA fragments in the sample may comprise counting the number of RNAs having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to lncRNA, and/or pgRNA, and/or the reference genome. The sequence data may be filtered to provide sequences having RNAs having 95%-99% sequence identity to lncRNA, and/or pgRNA, and/or the reference genome. The method may comprise detecting the amount of RNA fragments in the sample that comprises RNA sequences having 95%-99% sequence identity to lncRNA, and/or pgRNA, and/or the reference genome. The sequence data may be filtered to provide sequences having RNAs having 95%-100% sequence identity lncRNA, and/or pgRNA, and/or the reference genome. The method may comprise detecting the amount of RNA fragments in the sample that comprises RNA sequences having 95%-100% sequence identity to lncRNA, and/or pgRNA, and/or the reference genome. A person of ordinary skill in the art readily understands that any comparison of sequences is done in the context of sequences from the same organism as the sample, i.e., a human sample would be compared to a database or filters having human sequences.

The subject may be one that is predicted or determined to have a disease or non-disease state. The biological state classification may comprise a disease or non-disease state. The subject may be one that has a biological state. The subject may be one that is suspected of having a biological state. The subject may be one that has been diagnosed with a biological state. The subject may be one that has not been diagnosed with a biological state. The subject may be one that has one or more symptoms of a biological state. The biological state may comprise a disease state. The disease state may comprise a chronic disease. The disease state may comprise a neurological disease, cancer, a post-viral disease, or an autoimmune disease. The method may further comprise: identifying a treatment and/or treating the subject based on the predicted or determined biological or disease state classification. The methods may comprise treating the subject based on the detected RNAs. The biological state may also include a classification of “responder” or “non-responder” to a certain course of treatment.

The biological state may include or exclude recovered Covid, such as a subject that has had Covid in the past. The biological state may include or exclude Covid vaccinated, which denotes a subject that has received at least one Covid vaccine.

The disease state may comprise or exclude a neurological disease. The neurological disease may be Alzheimer's disease. The neurological disease may include or exclude mild cognitive impairment. The neurological disease may include or exclude Parkinson's disease. The neurological disease may include or exclude amyotrophic lateral sclerosis (ALS). The neurological disease may be multiple sclerosis (MS). MS may be further defined as relapsing-remitting MS (RRMS). The neurological disease may include or exclude multiple system atrophy (MSA). The neurological disease may include or exclude Creutzfeldt-Jakob Disease. The neurological disease may include or exclude depression. The neurological disease may include or exclude diabetes neuropathy. The neurological disease may include or exclude dementia. The neurological disease may include or exclude schizophrenia. The neurological disease may include or exclude progressive supranuclear palsy.

The disease state may comprise or exclude cancer. The cancer may comprise or exclude breast, head and neck, pancreatic, lung, prostate, colon, skin, uterine, liver, or brain cancer. The cancer may include or exclude a cancer described herein. The lung cancer may include or exclude non-small cell lung cancer. The disease state may comprise autoimmune disease. The autoimmune disease may comprise psoriatic arthritis. The post-viral disease may comprise Long Covid.

The treatment may comprise or exclude an rRNA-inhibiting/rRNA-interfering drug such as doxycycline. The treatment may comprise or exclude one or more of a cholinesterase inhibitor or NMDA receptor antagonist. The cholinesterase inhibitor may comprise or exclude one or more of donepezil, rivastigmine, and galantamine. The NMDA receptor antagonist may comprise or exclude memantine. The treatment may comprise or exclude one or more of levodopa, dopamine agonist, MAO-B inhibitor, COMT inhibitor, or deep brain stimulation. The treatment may comprise or exclude one or more of interferon beta-1a/b, glatiramer acetate, dimethyl fumarate, teriflunomide, fingolimod, natalizumab, ocrelizumab, corticosteroids, a muscle relaxant, antidepressant, anticonvulsant, plasmapheresis, therapeutic plasmapheresis exchange (TPE) or stem cell transplantation. The treatment may comprise or exclude non-drug treatments. The treatment may comprise or exclude an immunotherapy, chemotherapy, radiotherapy, surgery, or combinations thereof.

The subject may be one that is over 50 years in age. The subject may be one that is, is at least, or is at most 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or 101 years in age, or any derivable range therein.

The subject may have one or more symptoms of the disease state. The one or more symptoms may comprise involuntary movements, spasticity, tremors, spasms, involuntary contractions, visual symptoms, speech problems, cognitive decline, dementia, decrease in memory capacity, fainting, loss of consciousness, dizziness, and/or loss of strength or numbness of a limb.

The method may comprise determining the variance of the detected RNAs between a first biological state and a second biological state. The first biological state may comprise a disease state and the second biological state may comprise a non-disease state. The first biological state may comprise a first disease state and the second biological state may comprise a second disease state. The variance may be determined in a first, second, third, fourth, fifth, or nth disease state or biological state, wherein n is an integer between 1 and 100. Determining the variance may comprise training a machine learning classifier on a training set. The machine learning classifier may comprise or exclude one or more of Principal Component Analysis (PCA), Partial Least Squares Discriminant Analysis (PLS-DA), Sparse Partial Least Squares Discriminant Analysis, Multi-Dimensional Scaling (MDS), heatmap analysis, t-distributed Stochastic Neighbor Embedding (t-SNE), Generative Topographic Mapping (GTM), Self-Organizing Mapping (SOM), Linear Regression, Logistic Regression, Principal Component Regression, Linear Discriminant Analysis, Machine Learning, Deep Learning, Decision Trees, Random Forest, Neural Networks, Bayes Classifier, Support Vector Machines, Learning Vector Quantization, k-nearest Neighbors, Large Language Models, Parametric Models, Nonparametric Models, Quadratic Discriminant Analysis, Nearest Neighbor Algorithms, Combined Discriminant Analysis, k-means Clustering, Supervised Models, Unsupervised Models, Multivariable Regression Models, Penalized Multivariable Regression, Hierarchical Clustering, k-medians Clustering, Expectation-Maximization, Projection Pursuit, Mixture Discriminant Analysis, Flexible Discriminant Analysis, Uniform Manifold Approximation and Projection, Gradient Boosting, Ensemble Algorithms, Feature Selection Algorithms, or other types of models.

The machine learning classifier may include or exclude a continuously learning classifier. The machine learning classifier may include or exclude a static training classifier.

The method may comprise tuning the machine learning classifier. Tuning in machine learning refers to the process of optimizing the performance of a machine learning model by selecting the best values for its hyperparameters. Hyperparameters are parameters that are set by the user, and they control the behavior of the algorithm during training. Examples of hyperparameters include learning rates, regularization parameters, number of hidden layers in a neural network, and kernel types in support vector machines. There are various methods available for tuning hyperparameters, ranging from manual tuning to automated techniques. Manual tuning involves manually adjusting the hyperparameter values based on intuition, domain knowledge, and trial and error. Automated tuning methods can provide a more systematic and efficient way of tuning. These methods automate the process of searching for the best hyperparameter values by evaluating different combinations and selecting the ones that yield the best results. One commonly used automated tuning method is grid search. Grid search involves specifying a set of possible values for each hyperparameter and exhaustively searching through all possible combinations. It is a brute-force approach that can be computationally expensive, especially for large hyperparameter spaces. Random search is another popular tuning method that offers a more efficient alternative to grid search. Instead of exploring all possible combinations, random search samples random combinations of hyperparameters over a given search space. This approach is more effective at finding good hyperparameters and requires fewer evaluations than grid search. Bayesian optimization is a more advanced tuning method that uses probabilistic models to guide the search in the hyperparameter space. By leveraging the information gained from previous evaluations, Bayesian optimization can intelligently search for promising regions and quickly converge to the optimal hyperparameter values. The classifier may comprise a supervised model that has undergone tuning. The machine learning classifier may comprise reducing a dimensionality of the machine learning classifier based on a covariance of two or more parameters of the filtered sequence data of the training data set. The two or more parameters of the filtered sequence data of the training data set may be associated with two or more regions of interest of the filtered sequence data of the training data set. The two or more regions may comprise non-coding regions.

The parameter, ROIs, and/or features may include or exclude nucleic acid sequences. The parameter, ROIs, and/or features may include or exclude nucleic acid sequences that are non-coding. The one or more machine learning classifiers may be trained to predict, determine, or analyze the biological state classifications with at least a 95% accuracy based on the filtered sequence data of the training data set. The one or more machine learning classifiers may be trained to predict, determine, or analyze the biological state classifications with at least, at most, or about a 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) accuracy based on the filtered sequence data of the training data set. The one or more machine learning classifiers may be trained to predict, determine, or analyze the biological state classifications with at least 90% accuracy based on the filtered sequence data of the training data set. The one or more machine learning classifiers may be trained to predict, determine, or analyze the biological state classifications with 90-100% accuracy based on the filtered sequence data of the training data set.

The sample may comprise urine, fecal, blood, tears, cerebral spinal fluid, feces, or saliva sample. The sample may comprise a biological sample described herein. The sample from the subject may comprise a tissue sample, a blood sample, an oral sample, a saliva sample, a buccal sample, a whole blood sample, a fractionated sample, a plasma sample, a fecal sample, cerebral spinal fluid, tears, or a urine sample. The sample may comprise a blood sample from the subject. The sample may comprise cells. The sample may comprise plasma. The sample may comprise serum. The sample may comprise RNA.

The number of features used in the classifier to distinguish subjects of different biological states may be at least 100 or at most 300. The number of features used in the classifier to distinguish subjects of different biological states may be at least 2 or at most 10000000. The number of features used in the classifier to distinguish subjects of different biological states may be, be at least, or be at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 20000, 30000, 40000, 50000, 60000, 70000, 80000, 90000, 100000, 110000, 120000, 130000, 140000, 150000, 160000, 170000, 180000, 190000, 200000, 210000, 220000, 230000, 240000, 250000, 260000, 270000, 280000, 290000, 300000, 410000, 420000, 430000, 440000, 450000, 460000, 470000, 480000, 490000, 500000, 550000, 600000, 650000, 700000, 750000, 800000, 850000, 900000, 1000000, 2000000, 3000000, 4000000, 5000000, 6000000, 7000000, 8000000, 9000000, or 10000000 or any derivable range therein.

The number of samples used to train the machine learning classifier may be, be at least, or be at most 10, 100, 500, 1000, 1500, 2000, 2500, 3000, 3500, 4000, 4500, 5000, 6000, 7000, 8000, 9000, 10000, 11000, 12000, 13000, 14000, 15000, 16000, 17000, 18000, 19000, or 20000 different samples, or any derivable range therein.

Any method described herein can include or exclude at least 1, 2, 3, 4, 5, 6, 7, 8, 9, 10, or more (or any derivable range therein) of the following steps or methods: fragmentation of nucleic acids, end repair of nucleic acids, polyadenylation, cDNA synthesis, adapter addition, purification, library construction, library amplification, polymerase chain reaction, probe hybridization, elongation of nucleic acids, bead-mediated capture of nucleic acids, sequencing of nucleic acids, single-read sequencing, paired-end sequencing, barcode ligation of nucleic acids, multiplex sequencing, tagmentation, DNA and/or RNA library preparation, restriction digest, self-circularization of cDNAs, ligation of adaptors, PET (paired-end tags) library construction, RNA-PET, in vitro transcription, reverse transcription, high-throughput sequencing, RNA isolation, RNA selection, RNA depletion, polyA selection, rRNA depletion, RNA capture, and single cell RNA-Seq. A person of skill in the art is well aware of common techniques to accomplish each of the preceding steps.

Sequencing may comprise making a cDNA library of the RNA. Sequencing may comprise ligating adaptors to the cDNA and sequencing the cDNA. Depleting linear RNA may comprise depleting mRNA from the sample from the subject. The sequencing may comprise paired-end sequencing.

Detecting the amount of RNA fragments may comprise sequencing a biological sample from the subject comprising isolated RNA. Detecting the amount of RNA fragments may comprise or further comprise depleting linear RNA from the sample prior to sequencing. The subject may be one that was determined to have an increase or decrease in disease-associated RNA fragments by sequencing a biological sample from the subject comprising isolated RNA. The subject may be one that has been determined to have an increase or decrease in disease-associated RNA fragments by depleting linear RNA from the biological sample from the subject prior to sequencing.

The methods may be for preparing a sample for machine learning analysis. The methods may comprise or further comprise diagnosing, prognosing, or treating the subject based on the output of the biological state classification of the subject.

The kits of the disclosure may comprise a biological sample collection device. The collection device may comprise or exclude one or more reagents for stabilizing RNA and an exo-ribonuclease that preferentially hydrolyzes single-stranded linear RNA. The kits of the disclosure may comprise or exclude a PAXgene Blood RNA or HemaSure tube comprising one or more reagents for stabilizing RNA and an exoribonuclease that preferentially hydrolyzes single-stranded linear RNA. The exoribonuclease may be one that hydrolyzes RNA in the 3′-5′ direction. The exoribonuclease may comprise RNAse R. The kit may comprise ethylenediaminetetraacetic acid (EDTA). The kit may comprise RNA isolation reagents. The kit may comprise one or more of a resuspension buffer, a binding buffer, a wash buffer, an elution buffer, a proteinase, and a RNAse-free DNase. The proteinase may comprise Proteinase K. The DNase may comprise DNAse I. The one or more reagents may be RNAse-free. The kit may comprise a spin column.

The subject or patient may be a human subject or a human patient. The subject or patient may be a non-human animal. The non-human animal may be a cow/bull, bee, pig bat, monkey, camel, rat, mouse, rabbit, goat, chicken, bird, cat, dog. The subject may further be defined as a high risk subject. The subject may also be a plant, microbe, eukaryotic cell, or prokaryotic cell. The subject may be a mammal. The subject may be a non-human primate.

The methods may comprise treating the subject based on the detected RNAs. The methods may comprise or exclude treating the subject with an rRNA-inhibiting/rRNA-interfering drug such as doxycycline. The methods may comprise or exclude treating the subject with Aducanumab, Donepezil, Rivastigmine, Galantamine, Memantine, or combinations thereof. The methods may comprise or exclude treating the subject with corticosteroids, plasmapheresis, ocrelizumab, interferon beta, glatiramer acetate, fingolimod, dimethyl fumarate, diroximel fumarate, teriflunomide, siponimod, cladribine, natalizumab, alemtuzumab, or combinations thereof. The methods may comprise or exclude treating the subject with muscle relaxants, physical therapy, amantadine, modafinil, methylphenidate, dalfampridine, or combinations thereof. The methods may comprise or exclude treating the subject with one or more of Levodopa; a COMT inhibitor such as opicapone, entacapone, and/or tolcapone; a dopamine agonists such as bromocriptine, pergolide, pramipexole, ropinirole, piribedil, cabergoline, apomorphine, and/or lisuride; a MAO-B inhibitor such as safinamide, selegiline, and/or rasagiline; amantadine; anticholinergics; quetiapine; cholinesterase inhibitors; modafinil; pimavanserin, the Wahls protocol, or combinations thereof. In some aspects, the method comprises treating the subject with riluzole, edaravone, gabapentin, pregabalin, tricyclic antidepressants, nonsteroidal anti-inflammatory drugs, opioids, selective serotonin reuptake inhibitors, benzodiazepines, baclofen, tizanidine, atropine, scopolamine, amitriptyline, glycopyrrolate, mexiletine, sodium phenylbutyrate, taurursodiol, or combinations thereof. The methods may comprise or exclude treating the subject with non-drug treatments.

Methods described herein include, but are not limited to, methods for treating a subject, methods for determining a biological state classification of a subject, methods for evaluating a biological state classification of a subject, methods for evaluating a subject for a biological state classification more than once, methods of diagnosing a subject, methods for evaluating non-linear RNA sequence data from a biological sample from a subject, methods for evaluating a a subject for a disease or condition, methods for diagnosing a subject for a disease or condition, methods for prognosing a subject for a disease or condition, methods for monitoring a subject for a disease or condition, methods for evaluating lncRNA and/or pgRNA in a biological sample, methods of generating a sample enriched in non-coding RNA for using in inputting sequence data into a machine learning classifier, methods of applying sequence data to a machine learning classifier, methods for training a machine learning classifier, a method for evaluating a subject for multiple diseases and conditions, methods for evaluating whether EANS sequence data can be used for evaluating a subject for a biological state classification or specific disease or condition comprising or comprising at least, or comprising at most 1, 2, 3, 4, 5, 6 or more of the following steps; obtaining a biological sample from a subject, obtaining a sample from a subject comprising RNA, enriching for noncoding RNA in a sample from a subject, reducing the amount of coding RNA in a sample, eliminating coding RNA in a sample, destroying coding RNA in a sample using exonuclease, discarding coding RNA from a sample, enriching for nonlinear RNA in a sample, sequencing nonlinear RNA in a sample, sequencing fragments of nonlinear RNA in a sample, contacting a biological sample with an exonuclease, contacting a biological sample with RNase R under condition to destroy linear RNA, sequencing nonlinear RNA, sequencing RNA in a sample depleted of nonlinear RNA, obtaining sequence data about nonlinear RNA in a sample, detecting the amount of RNA fragments in a sample, obtaining sequence data about lncRNA and/or pgRNA and/or a reference genome, applying sequence data to a machine learning classifier, filtering sequence data, outputting biological state classification, determining a subject has a disease or condition, monitoring the subject for a disease or condition, preparing a report identifying the patient as having or not having a disease or condition, determining parameters, ROIS, features and/or filters for a machine learning classifier, and/or tuning a machine learning classifier.

The term “training data,” as used herein generally refers to data that can be input into models, statistical models, algorithms and any system or process able to use existing data to make predictions.

As used herein, a “model” may include one or more algorithms, one or more mathematical techniques, one or more machine learning algorithms, or a combination thereof.

As used herein, “machine learning” may be the practice of using algorithms to parse data, learn from it, and then make a determination or prediction about something in the world. Machine learning uses algorithms that can learn from data without relying on rules-based programming.

As used herein, a “parameter” may refer to an attribute of an observable phenomenon for which a quantitative value can be assigned assessing a magnitude of, a presence of, and/or an absence of the attribute. For example, when used in “parameters for the sequence profile,” the “parameters” may refer to attributes of the sequence profile, such as but not limited to regions of interest (ROIs) for the sequence profile. Thus, if the attributes for the sequence profile corresponds to regions of interest (ROIs), a quantitative value assigned to the parameter can assess a presence or absence of the ROI in the sequence profile, a level of expression for the ROI, and/or a level of expression of the ROI that is above a predetermined threshold.

As used herein, a “feature vector” may include an ordered list of quantitative values respectively assessing a set of parameters. Each position in the ordered list may be associated with a respective parameter, such that a value in a given position may assess the parameter associated with the given position. For example, a feature vector may comprise an ordered list of quantitative values indicating levels of expression for a respective set of ROIs for a sequence profile.

The references to the methods of treatment by therapy or surgery or in vivo diagnosis methods in example 1 of this description and in the claims and disclosure of this description are to be interpreted as references to compounds, pharmaceutical compositions and medicaments of the present invention for use in those methods.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of”

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-1B are schematic diagrams of an exemplary workflows for the detection of RNA sequences associated with a neurological disorder.

FIG. 2. Exon Reduction after Removing linear RNA.

FIG. 3A-3B. Shown is the percent small RNA (FIG. 3A, top) and the percent non-repetitive RNA (FIG. 3A, bottom) in apparently healthy normal samples filtered by the lncRNA database. FIG. 3B shows the percent small RNA (FIG. 3B, top) and the percent non-repetitive RNA (FIG. 3B, bottom) in apparently healthy normal samples filtered by the pgRNA database.

FIG. 4A-4F. Shows that different Bioinformatics/Machine Learning Tools can measure clusters in RNA samples depleted of linear RNA. FIG. 4A is a sparse partial Least Squares Discriminant Analysis filtered against the HG38 reference genome. FIG. 4B is a Principal Component Analysis (95% confidence) filtered against the HG38 reference genome.

FIG. 4C is a sparse partial Least Squares Discriminant Analysis filtered against the pgRNA panel. FIG. 4D is a Principal Component Analysis filtered against the pgRNA panel; FIG. 4E is a sparse partial Least Squares Discriminant Analysis filtered against the lncRNA panel. FIG. 4F is a Principal Component Analysis filtered against the lncRNA panel. N=untreated with enzyme digestion; R=enzyme-treated sample.

FIG. 5A-5B shows a sPLS-DA plot of different disease states data filtered on EANS 1 (lncRNA—FIG. 5A). and EANS 2 (pgRNA—FIG. 5B). Shown are apparently healthy normal (AHN) and three different diseases.

FIG. 6 shows the notable temporal pattern of eansRNA expression as a result of steroid treatment.

FIG. 7A-7B depict the Principle Component 1 (PC1) changes from lncRNA EANS over time in two patients with MCI who underwent Therapeutic Plasma Exchange (TPE) therapy. Patient 19813 (top) represents a typical example observed in patients with Mild Cognitive Impairment (MCI) undergoing TPE, showing a significant change in variance occurring around day 28. In contrast, patient 19914 (bottom) illustrates an individual whose PC1 variance remained unchanged, ultimately revealing a misdiagnosis of MCI.

FIG. 8A-8C are Venn diagrams that show three distinct populations of eansRNA “TPE non-responders,” “TPE negative responders/recovered,” and “TPE novel responders” associated with three patients undergoing therapeutic plasma exchange (TPE). Following the procedure, 7212, 5196, and 5994 eansRNA sequences were identified for patients 2, 3, and 4, respectively. While most sequences were unique to each patient, some overlapped with those from patients with mild cognitive impairment (MCI).

FIG. 9A-9E shows the cluster analysis of patient F363. FIG. 9A: patient F363 was found to cluster with Parkinson's disease. FIG. 9B: patient F363 did not cluster with mild cognitive impairment. FIG. 9C: patient F363 did not cluster with multiple sclerosis. FIG. 9D: shows that patient F363 also did not cluster with cancer (clustered with apparently healthy normal (AHN). FIG. 9E: Heat Map Analysis. The second method of querying the data is to present the results as a heat map of the patient's sample against a selected disease database. Data from seven PD patients and five AHN controls were grouped by expression profiles next to the patient's data.

FIG. 10 illustrates the longitudinal assessment of a relapse remitting multiple sclerosis patient responding to therapeutic interventions. Data points represent PC1 variance measured at multiple time points, highlighting changes in the patient's progression and adaptation to treatment.

FIG. 11A is a block diagram of a computer system in accordance with various embodiments.

FIG. 11B is a block diagram illustrating an example process for classifying biological states in accordance with various non-limiting embodiments of the present disclosure.

### DETAILED DESCRIPTION OF THE INVENTION

### I. Overview of Exemplary Workflow

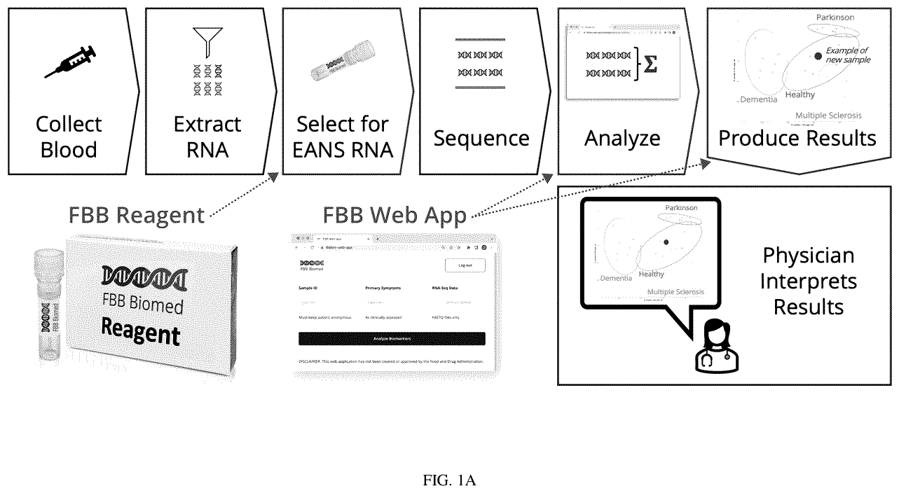

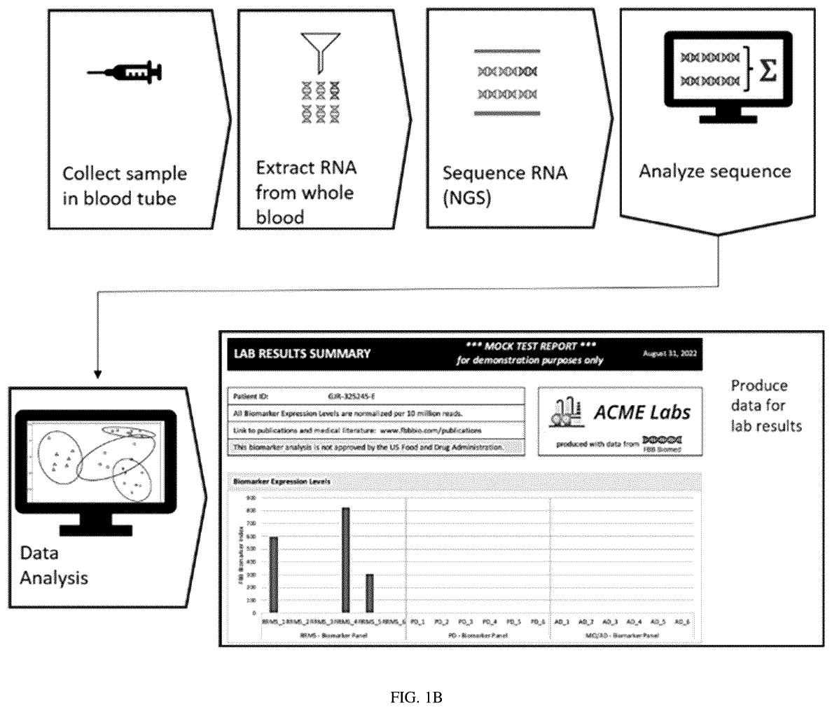

FIG. 1 is a schematic diagram of an exemplary workflow for the detection of RNA sequences associated with a neurological disorder for use in diagnosis and/or treatment in accordance with one or more methods of the disclosure. Workflow may include various operations including, for example, sample collection, sample preparation and processing, data analysis, and output generation.

### A. Sample Collection

Sample collection may include, for example, obtaining a biological sample of one or more subjects. Biological sample may take the form of a specimen obtained via one or more sampling methods. Biological sample may be representative of subject as a whole or of a specific tissue, cell type, or other category or sub-category of interest. Biological sample may be obtained in any of a number of different ways. In various embodiments, biological sample includes whole blood sample obtained via a blood draw. In other embodiments, biological sample includes set of aliquoted samples that includes, for example, a serum sample, a plasma sample, a blood cell (e.g., white blood cell (WBC), red blood cell (RBC) sample, another type of sample, or a combination thereof. Biological samples may include or exclude nucleic acids (e.g., ssDNA, dsDNA, RNA), organelles, amino acids, peptides, proteins, carbohydrates, glycoproteins, or any combination thereof.

### B. Sample Preparation

In certain aspects, methods involve a sample that has been obtained from a subject. The sample may be obtained by methods including biopsy, such as fine needle aspiration, core needle biopsy, vacuum assisted biopsy, incisional biopsy, excisional biopsy, punch biopsy, shave biopsy or skin biopsy. The sample may be obtained from any source including but not limited to blood, serum, plasma, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be obtained by methods known in the art. In certain embodiments the samples are obtained by biopsy. In other embodiments the sample is obtained by swabbing, endoscopy, scraping, phlebotomy, or any other methods known in the art. In some cases, the sample may be obtained, stored, or transported using components of a kit of the present methods. In some cases, multiple samples, such as multiple plasma or serum samples may be obtained for diagnosis by the methods described herein. In other cases, multiple samples, such as one or more samples from one tissue type (for example ovaries or related tissues) and one or more samples from another specimen (for example serum) may be obtained for diagnosis by the methods. Samples may be obtained at different times, stored, and/or analyzed by different methods. For example, a sample may be obtained and analyzed by routine staining methods or any other cytological analysis methods.

In some embodiments the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, blood draw, endoscopy, or phlebotomy. The method of needle aspiration may further include fine needle aspiration, core needle biopsy, vacuum assisted biopsy, or large core biopsy. In some embodiments, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods.

In some embodiments of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some embodiments of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, gene fusions, gene chimeras, expression products, gene expression products, or gene expression product fragments of an individual to be tested. Methods for determining sample suitability and/or adequacy are provided.

In some embodiments, the subject may be referred to a specialist such as a neurologist oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

In various embodiments, a single run can analyze a sample (e.g., the sample including RNA), an external standard, and an internal standard. In various embodiments, external standards may be analyzed prior to analyzing samples. In various embodiments, the external standards can be run independently between the samples. In some embodiments, external standards can be analyzed after every 1, 2, 3, 4, 5, 6, 7, 8, 9, 10, 11, 12, 13, 14, 15, 16, 17, 18, 19, 20, or more experiments. In various embodiments, external standard data can be used in some or all of the normalization systems and methods described herein.

Sample preparation and processing may include, for example, one or more operations to form RNA preparations. Embodiments may include depletion of linear RNA by methods described herein, for example, by contacting the RNA preparation with RNAse R. Various protocols known in the art can be employed to generate samples for use with one or more of the embodiments described herein. A sample (e.g., suspension) can be generated from any type of cells. For example, such cells may include eukaryotic cells (e.g., eukaryotic cells with a chromatin structure). Further, cells from fresh or cryopreserved cell lines (e.g., human cell lines, mouse cell lines, etc.), as well as more fragile primary cells, may be used. In one or more embodiments, the cells in a sample include, but are not limited to, immune cells (e.g., B cells, T cells), peripheral blood mononuclear cells (PBMCs), bone marrow mononuclear cells (BMMCs), lymphocytes, or a combination thereof. Still further, the sample may be formed by cells from a single donor or multiple donors.

The samples may be further processed by construction of a library from the sample of RNA. Library construction includes the generation of a library. In one or more embodiments, library contains a plurality of DNA fragments. These DNA fragments may be utilized for sequencing. In one or more embodiments, cDNA molecules can be used as templates for PCR to produce a library. Library may include molecules from one or more samples, molecules from samples from one or more donors, molecules from multiple libraries corresponding to one or more donors, or a combination thereof.

In one or more embodiments, library construction includes library preparation. Library preparation may include, for example, adding one or more adapter sequences, optionally a sample index (SI) sequence, or a combination thereof to each of the recovered barcoded cDNA molecules in library construction. An SI sequence may include, for example, without limitation, one or more oligonucleotides (e.g., four oligonucleotides) that enable unique identification of the original sample.

### C. Sequencing

In some embodiments, RNA may be analyzed and/or sequenced. Methods disclosed herein include measuring expression of RNAs (RNAs) such as pseudogene RNAs (pgRNA) and long noncoding RNAs (lncRNAs). Measurement of expression can be done by a number of processes known in the art. The process of measuring expression may begin by extracting RNA from a biological sample. Extracted RNA can be detected by hybridization (for example by means of Northern blot analysis or DNA or RNA arrays (microarrays) after converting RNA into labeled cDNA) and/or amplification by means of a enzymatic chain reaction. Quantitative or semi-quantitative enzymatic amplification methods such as polymerase chain reaction (PCR) or quantitative real-time RT-PCR or semi-quantitative RT-PCR techniques can be used. Suitable primers for amplification methods encompassed herein can be readily designed by a person skilled in the art. Other amplification methods include ligase chain reaction (LCR), transcription-mediated amplification (TMA), strand displacement amplification (SDA), isothermal amplification of nucleic acids, and nucleic acid sequence based amplification (NASBA). Expression levels of RNAs may also be measured by RNA sequencing methods known in the art. RNA sequencing methods may include mRNA-seq, total RNA-seq, long range sequencing (commercial kits available by PacBio, for example), targeted RNA-seq, small RNA-seq, single-cell RNA-seq, ultra-low-input RNA-seq, RNA exome capture sequencing, and ribosome profiling. Sequencing data may be processed and aligned using methods known in the art. In some embodiments, sequencing may be performed to generate approximately 10M, 15M, 20M, 25M, 30M, 35M, 40M or more reads. The reads may include paired reads. The sequencing may be performed at a read length of approximately 10 bp, 15 bp, 20 bp, 25 bp, 30 bp, 35 bp, 40 bp, 45 bp, 50 bp, 55 bp, 60 bp, 65 bp, 70 bp, 75 bp, 80 bp, 85 bp, 90 bp, 95 bp, 100 bp, 105 bp, 110 bp, or longer.

To normalize the expression values of one RNA region of interest (ROI), such as pgRNA or lncRNA, among different samples, comparing the ROI in the samples from the subject object of study with a control RNA level is possible. As it is used herein, a “control RNA” is an RNA of a ROI for which the expression level does not differ among different non-diseased individuals. In some aspects, the ROI may be constitutively expressed in all types of cells. A control RNA is preferably a RNA derived from a constitutively expressed RNA. A known amount of a control RNA may be added to the sample(s) and the value measured for the level of the ROI may be normalized to the value measured for the known amount of the control RNA. Normalization for some methods, such as for sequencing, may comprise calculating the reads per kilobase of transcript per million mapped reads (RPKM) for a ROI, or may comprise calculating the fragments per kilobase of transcript per million mapped reads (FPKM) for a ROT. Normalization methods may comprise calculating the log 2-transformed count per million (log-CPM). It can be appreciated to one skilled in the art that any method of normalization that accurately calculates the expression value of an RNA for comparison between samples may be used.

Methods disclosed herein may include comparing a measured expression level to a reference expression level. The term “reference expression level” refers to a value used as a reference for the values/data obtained from samples obtained from patients. The reference level can be an absolute value, a relative value, a value which has an upper and/or lower limit, a series of values, an average value, a median, a mean value, or a value expressed by reference to a control or reference value. A reference level can be based on the value obtained from an individual sample, such as, for example, a value obtained from a sample from the subject object of study but obtained at a previous point in time. The reference level can be based on a high number of samples, such as the levels obtained in a cohort of subjects having a particular characteristic. The reference level may be defined as the mean level of the patients in the cohort. The reference may be from subjects that are healthy, subjects without one or more neurological disorder(s), subjects that are age-matched, subjects that are gender-matched, and/or subjects that are race-matched. A reference level can be based on the expression levels of the markers to be compared obtained from samples from subjects who do not have a disease state or a particular phenotype. The person skilled in the art will see that the particular reference expression level can vary depending on the specific method to be performed.

Some embodiments include determining that a measured expression level is higher than, lower than, increased relative to, decreased relative to, equal to, or within a predetermined amount of a reference expression level. In some embodiments, a higher, lower, increased, or decreased expression level is, is at least, or is at most 1, 1.5, 2, 2.5, 3, 3.5, 4, 4.5, 5, 5.5, 6, 7, 8, 9, 10, 12, 14, 16, 18, 20, 50, 100, 150, 200, 250, 500, or 1000 fold (or any derivable range therein) or at least 10, 20, 30, 40, 50, 60, 70, 80, 90, 100, 200, 300, 400, 500, 600, 700, 800, or 900% different than the reference level, or any derivable range therein. These values may represent a predetermined threshold level, and some embodiments include determining that the measured expression level is higher by a predetermined amount or lower by a predetermined amount than a reference level. In some embodiments, a level of expression may be qualified as “low” or “high,” which indicates the patient expresses a certain ROI or RNA at a level relative to a reference level or a level with a range of reference levels that are determined from multiple samples meeting particular criteria. The level or range of levels in multiple control samples is an example of this. In some embodiments, that certain level or a predetermined threshold value is at, below, or above 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percentile, or any range derivable therein. Moreover, a threshold level may be derived from a cohort of individuals meeting a particular criterion or set of criteria. The number in the cohort may be, be at least, or be at most 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 700, 800, 900, 1000, 1100, 1200, 1300, 1400, 1500, 1600, 1700, 1800, 1900, 2000 or more (or any range derivable therein). A measured expression level can be considered equal to a reference expression level if it is within a certain amount of the reference expression level, and such amount may be an amount that is predetermined. The predetermined amount may be within 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 18, 19, 20, 21, 22, 23, 24, 25, 30, 35, 40, 45, or 50% of the reference level, or any range derivable therein.

For any comparison of RNA expression levels to a mean expression level or a reference expression level, the comparison is to be made on a ROI-by-ROI and RNA-by-RNA basis.

### D. Sequence Filter

The National Human Genome Research Institute (NHGRI) launched a public research consortium named ENCODE, the Encyclopedia Of DNA Elements, in September 2003, to carry out a project to identify all functional elements in the human genome sequence. After a successful pilot phase on 1% of the genome, the scale-up to the entire genome was conducted. The annotated database can be found on the world wide web at gencodegenes.org. This database contains a comprehensive gene annotation of long non-coding RNA (lncRNAs) on the reference chromosomes. This database also contains an annotated sequences of predicted pseudogene (pgRNA) on reference chromosomes. The lncRNAs database includes generic long non-coding RNA biotype that replaced the following biotypes: 3prime_overlapping_ncRNA, antisense, bidirectional_promoter_lncRNA, lincRNA, macro_lncRNA, non_coding, processed_transcript, sense_intronic and sense_overlapping. Pseudogenes included in the pg database include genes that have homology to proteins but generally suffer from a disrupted coding sequence and an active homologous gene can be found at another locus, sometimes as a retropseudogene. The pgRNA entries may have an intact coding sequence or an open but truncated ORF, in which case there is other evidence used (for example genomic polyA stretches at the Y end) to classify them as a pseudogene. Pseudogenes included are those i) that lack introns and are thought to arise from reverse transcription of mRNA followed by reinsertion of DNA into the genome; ii) owing to a SNP/DIP but in other individuals/haplotypes/strains the gene is translated; iii) owing to a reverse transcribed and re-inserted sequence; iv) where protein homology or genomic structure indicates a pseudogene, but the presence of locus-specific transcripts indicates expression; v) that has mass spec data suggesting that it is also translated; vi) without a parent gene, as it has an active orthologue in another species; and vii) that can contain introns since produced by gene duplication. Besides ENCODE, other alignment references can be used to the same effect, for example the Ensembl reference for Homo Sapiens.

The sequence may be analyzed, filtered, and/or compared to a reference genome. A reference genome is a digital nucleic acid sequence database that is a representative example of genomic sequences and/or the set of genes in an organism of a species. For example, the human reference genome assembly, GRCh38/hg38, is derived from over 60 genomic clone libraries prepared from human biological samples. There are reference genomes for multiple species of viruses, bacteria, fungus, plants, and animals. Reference genomes can be assessed online at several locations. For example, the Ensemble or USCS Genome browser can be used.

The Ensemble genome database is available on the world wide web at ensemble.org. Similar databases and browsers are found at National Center for Biotechnology Information (NCBI) (accessed on the web at ncbi.nlm.nih.gov) and the University of California, Santa Cruz (UCSC) (the UCSC genome browser may be accessed online at genome.ucsc.edu. The reference genome may be one that includes non-coding RNA. The reference genome may be one that includes double-stranded RNA.

The sequenced RNA can be filtered, which indicates that sequences that do not align or align with at least 90, 91, 92, 93, 94, 95, 96, 97, 98, or 99 percent sequence identity, are removed from the analysis.

### E. Data Analysis

Data analysis includes processing and analyzing the filtered sequences. This analysis may be performed in any number of different ways to extract various pieces of information from the sequence dataset. Various methods and systems may be employed to analyze the sequence dataset received as input in accordance with one or more embodiments described herein.

In one or more embodiments, data analysis may be implemented using hardware, software, firmware, or a combination thereof. For example, data analysis may be implemented using a computing platform. Computing platform may include a computer system, a cloud computing platform, some other type of computing platform, or a combination thereof. The computer system may include a single computer or multiple computers in communication with each other.

In one or more embodiments, computing platform is communicatively coupled (e.g., via direct wired connection(s) or wireless connection(s)) to data store, display system, set of input devices, or a combination thereof. In one or more embodiments, display system, one or more input devices of set of input devices, or both are at least partially integrated within computing platform. In other embodiments, display system, one or more input devices of set of input devices, or a combination thereof may be separate from but in communication with computing platform. Computing platform may receive, retrieve, or otherwise obtain the sequence dataset from a data store. Display system may be used to, for example, without limitation, visualize sequence dataset, information generated via data analysis, or both. Set of input devices enable a user to provide user input for utilization during data analysis. Any combination or configuration of computing platform, data store, display system, or set of input devices may be integrated into a system assembly (e.g., housed in a same housing and/or communicatively coupled via conventional device/component connection means).

In one or more embodiments, data analysis includes a machine learning system, which may itself be comprised of any number of machine learning models and/or algorithms. For example, a machine learning system may include, but is not limited to, at least one of Principal Component Analysis (PCA), Partial Least Squares Discriminant Analysis (PLS-DA), Sparse Partial Least Squares Discriminant Analysis, Multi-Dimensional Scaling (MDS), heatmap analysis, t-distributed Stochastic Neighbor Embedding (t-SNE), Generative Topographic Mapping (GTM), Self-Organizing Mapping (SOM), Linear Regression, Logistic Regression, Principal Component Regression, Linear Discriminant Analysis, Machine Learning, Deep Learning, Decision Trees, Random Forest, Neural Networks, Bayes Classifier, Support Vector Machines, Learning Vector Quantization, k-nearest Neighbors, Large Language Models, Parametric Models, Nonparametric Models, Quadratic Discriminant Analysis, Nearest Neighbor Algorithms, Combined Discriminant Analysis, k-means Clustering, Supervised Models, Unsupervised Models, Multivariable Regression Models, Penalized Multivariable Regression, Hierarchical Clustering, k-medians Clustering, Expectation-Maximization, Projection Pursuit, Mixture Discriminant Analysis, Flexible Discriminant Analysis, Uniform Manifold Approximation and Projection, Gradient Boosting, Ensemble Algorithms, Feature Selection Algorithms, or other types of models. In various embodiments, model includes a machine learning model that comprises any number of or combination of the models or algorithms described above.

In various embodiments, the biological state classifier comprises a probability that the biological sample is positive for the biological state and the machine learning model may be configured to generate an output that identifies the biological sample as either evidencing (“positive for”) the biological state when the classifier is greater than a selected threshold or not evidencing (“negative for”) the biological state when the classifier is not greater than the selected threshold. In various embodiments, the machine learning model is a machine learning model that is trained to determine weight coefficients for a panel of regions of interest (ROIs), such as pgRNA and/or lncRNA.

### F. Computer Implemented System

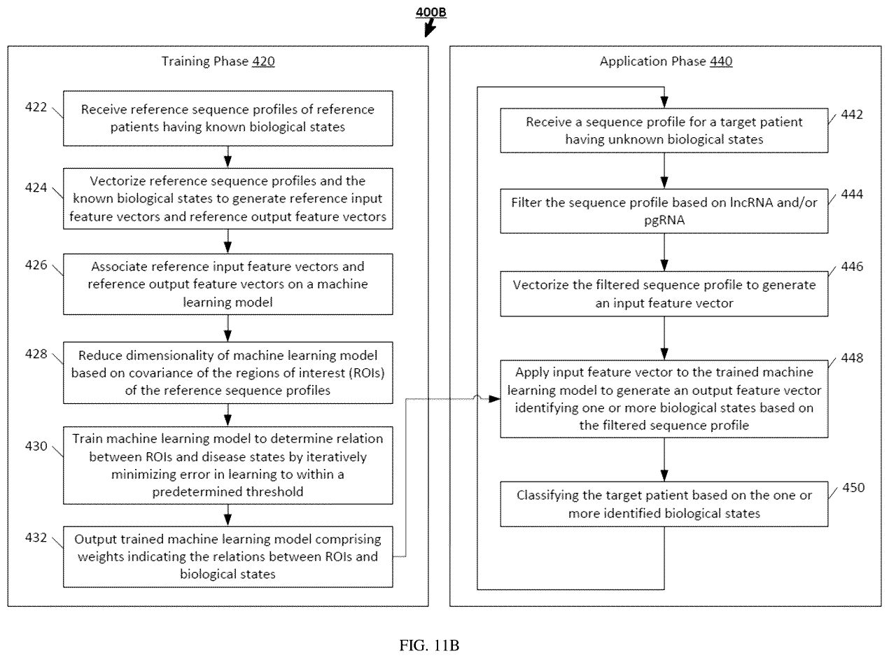

FIG. 11 is a block diagram of a computer system in accordance with various embodiments. Computer system 400A may be an example of one implementation for computing platform described above.

In one or more examples, computer system 400A can include a bus 402 or other communication mechanism for communicating information, and a processor 404 coupled with bus 402 for processing information. In various embodiments, computer system 400A can also include a memory, which can be a random-access memory (RAM) 406 or other dynamic storage device, coupled to bus 402 for determining instructions to be executed by processor 404. Memory also can be used for storing temporary variables or other intermediate information during execution of instructions to be executed by processor 404. In various embodiments, computer system X00 can further include a read only memory (ROM) 408 or other static storage device coupled to bus 402 for storing static information and instructions for processor 404. A storage device 410, such as a magnetic disk or optical disk, can be provided and coupled to bus 402 for storing information and instructions.

In various embodiments, computer system 400A can be coupled via bus 402 to a display 412, such as a cathode ray tube (CRT), liquid crystal display (LCD), or light emitting diode (LED) for displaying information to a computer user. An input device 414, including alphanumeric and other keys, can be coupled to bus 402 for communicating information and command selections to processor 404. Another type of user input device is a cursor control 416, such as a mouse, a joystick, a trackball, a gesture input device, a gaze-based input device, or cursor direction keys for communicating direction information and command selections to processor 404 and for controlling cursor movement on display 412. This input device 414 typically has two degrees of freedom in two axes, a first axis (e.g., x) and a second axis (e.g., y), that allows the device to specify positions in a plane. However, it should be understood that input devices 414 allowing for three-dimensional (e.g., x, y, and z) cursor movement are also contemplated herein.

Consistent with certain implementations of the present teachings, results can be provided by computer system 400A in response to processor 404 executing one or more sequences of one or more instructions contained in RAM 406. Such instructions can be read into RAM 406 from another computer-readable medium or computer-readable storage medium, such as storage device 410. Execution of the sequences of instructions contained in RAM 406 can cause processor 404 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 404 for execution. Such a medium can take many forms, including but not limited to, non-volatile media, volatile media, and transmission media. Examples of non-volatile media can include, but are not limited to, optical, solid state, magnetic disks, such as storage device 410. Examples of volatile media can include, but are not limited to, dynamic memory, such as RAM 406. Examples of transmission media can include, but are not limited to, coaxial cables, copper wire, and fiber optics, including the wires that comprise bus 402.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 404 of computer system 400A for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 400A as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 400, whereby processor 404 would execute the analyses and determinations provided by these engines, subject to instructions provided by any one of, or a combination of, the memory components RAM 406, ROM, 408, or storage device X10 and user input provided via input device 414.

### G. Example Training and Application of Machine Learning Model

FIG. 11B is a block diagram illustrating an example process 400B for classifying biological states, according to non-limiting embodiments of the present disclosure. As illustrated, process 400B includes a number of enumerated steps, but aspects of process 400B may include additional steps before, after, and in between the enumerated steps. In some embodiments, one or more of the enumerated steps may be omitted or performed in a different order. Process 400B, which may comprise a training phase 420 and an application phase 440, may be performed by one or more computer systems (e.g., such as but not limited to computer system 400A). For example, process 400B may be performed by one or more processors (such as, but not limited to, processor 404) based on computer-executable or machine readable instructions stored in a memory (such as, but not limited to, storage device 410) of the one or more computer systems. In some aspects, one or more blocks of the training phase 420 may be performed by a computer system separate or distinct from the computer system performing one or more blocks of the application phase 440, for example, to conserve computer resources and/or bandwidth.

In various embodiments, the training phase 420 may involve receiving reference sequence profiles of reference patients having known biological states (block 422). For example, a sequence profile may be received for each of a plurality of reference patients. As used herein, the reference patient may refer to a patient for whom the one or more biological states may already be known and from which a reference sequence profile may be used for training a machine learning model. For example, the known biological state may be an indication of, no disease state (e.g., the reference patient is an AHN patient), a first disease state, a second disease state, etc. Each reference sequence profile may correspond to at least a portion of the sequenced RNA of the respective patient. In some embodiments, each reference sequence profile may correspond to a filtered sequence of the sequenced RNA of the respective reference patient. The filtering may be based on identified lncRNA and/or pgRNA of the respective patient. Furthermore, each reference sequence profile may include one or more regions of interest (ROI) that may be relevant for the known biological states. In some embodiments, each of or at least a subset of the received reference sequence profiles may be labeled with or otherwise associated with the known biological states (e.g., for subsequent supervised learning). In some embodiments, unlabeled reference sequence profiles may be used for validation and testing of the machine learning model during the training in order to improve the accuracy and robustness of machine learning model.

The machine learning model trained in training phase 420 (which may then be applied in application phase 440) may itself be comprised of any number of or combination of machine learning models and/or algorithms previously discussed. Furthermore, the machine learning models trained in training phase 420 may be specifically trained to determine one or more biological states from a sequence profile.

In some aspects, the reference sequence profiles may be unstructured and a processor (e.g., a natural language processor, image processor, a special purpose gene sequencing processor, etc.) may process, translate, decrypt, decipher, and/or quantify the unstructured data into a format that can be vectorized.

At block 424, the computer system may vectorize the reference sequence profiles and the known biological states to generate reference input feature vectors and reference output feature vectors, respectively. For example, the computer system may process unstructured data comprising the sequence profiles to quantify, as feature vectors, relevant information for the sequence profiles. Such relevant information may include one or more values for each of set of parameters for the sequence profile. In some embodiments, the set of parameters may correspond to a plurality of ROIs of the sequence profile. Thus, the values may correspond to a level of expression for each of the ROIs of the sequence profile. Also or alternatively, the values may correspond to a level of expression of the ROI that is above a predetermined threshold, where the predetermined threshold may be based on a reference expression level for the ROI.

At block 426, each reference input feature vector may be associated with a respective reference biological state of the respective reference patient. Thus, each reference input feature vector may be paired with a respective reference output feature vector. For example, the reference input feature vector and the respective reference biological state may be written into a linked data structure in a memory device (e.g., the electronic storage device 410, RAM 406, and/or ROM) of computer system 400A.

At block 428, the computer system may reduce the dimensionality of the machine learning model based on the covariance of the parameters (e.g., ROIs) of the reference sequence profiles. For example, of the ROIs in a reference sequence profiles, the computing system may detect (e.g., based on principal component analysis (PCA) or a similar technique) that two or more ROIs may be correlated to an extent that it may be more efficient for the computing system to treat the two or more ROIs as a single parameter. In some embodiments, the dimensionality reduction may be performed as part of the vectorization process. For example, the vectorization may involve the computer system 400A compressing unstructured data received in block 422 such that disparate inputs for a given parameter may be aggregated as a composite input for that parameter. The vectorization may result in a reference input feature vector comprising composite data inputs for each of a plurality of input parameters. Also or alternatively, the dimensionality reduction may be performed after vectorization using the paired reference feature vectors, for example, via techniques for determining co-variance, such as principal component analysis (PCA). Thus, dimensionality reduction may allow redundant or unnecessary parameters to be removed, for example, from the reference input feature vector. The dimensionality reduction may enhance the speed of the machine learning model being trained or may be used to overcome issues of overfitting.

At block 430, the computer system may train the machine learning model to determine (e.g., learn) relations between parameters (e.g., ROIs) and disease states. The training may involve iteratively minimizing error in the learning to within a predetermined threshold (e.g., tolerance level). For example, for each pair of reference input feature vector (representing relevant ROIs of the reference sequence profiles) and reference output feature vector (representing the known biological states), the input feature vector may be inputted within the machine learning model with randomized or initialized weights and/or biases for each input parameter represented by the reference input feature vector. The machine learning model may be structured to allow the weights to be iteratively adjusted through an error minimization process as the relation between the reference input feature vector and the respective reference output feature vector is determined. In some embodiments, the iterative error minimization process may continue until the error rate are below about a 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%. 0.6%, or 0.5%, or below an error rate based any range derivable therein. For example, the iterative error minimization process may continue until the error rate is below about 5% (e.g., preferably below about 1%). Also or alternatively, the iterative error minimization process may continue until the machine learning model (e.g., machine learning classifier) is trained to predict, determine, or analyze the disease states (e.g., correctly) with at least about 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% accuracy. For example, the iterative error minimization process may continue until the machine learning model is trained to predict, determine, or analyze the disease states with at least about 95% accuracy (e.g., preferably at least about 99% accuracy). In some embodiments, the number of iterations and/or the number of parameters involved in the machine learning model may be at large scales, rendering such processes as impractical to be performed outside of a computer system environment. For example, in some embodiments, the processor 404 may be equipped or configured to perform the large number of iterations at a sufficient speed to avoid causing latency in other processes of the computer system 400A.

In some embodiments, the learning of the relations may be based on determining which parameters (e.g., ROIs) are relevant and to what level of expression for each biological state. In some embodiments, such learning may be performed by way of a classification model where the machine learning model learns to classify an input feature vector indicating the ROIs expressed and their level of expression for a respective sequence profile into one of a plurality of different biological states. In some embodiments, the classification may be based on the distance of the input feature vector (e.g., based on the quantification of parameters of the input feature vector) to other input feature vectors.

The relations between the parameters (e.g., ROIs) and the biological states may be represented by the set of weights assigned to the parameters (e.g., ROIs of the sequence profile and their level of expression) represented by the input feature vector. The initial set of weights for the parameters of the input feature vector may be tested for how correctly the set of weights attribute the significance of various parameters in their ability to predict the classification of the biological state of the patient represented by the output feature vector. Each determined classification may be a quantitative and/or binary data that is compared to the known data for the one or more biological states. If the difference does not fall below a predetermined threshold or tolerance, an iterative process occurs involving a new set of weights for the parameters. The training involves determining a correct set of weights for the input parameters of the input feature vector.

At block 432, the computer system may output the trained machine learning model comprising the finalized set of weights indicating relations between parameters (e.g., ROIs) and biological states. For example, the trained machine learning model may be stored in a memory (e.g., stored device 410 of computer system 400A) or may otherwise may accessible to the computer system that performed the training or to another computer system. Also or alternatively, the trained machine learning model may be stored in a local or remote server that may be accessed by a computer system performing one or more blocks of the application phase 440.

In various embodiments, the application phase 440 may involve a computer system having a processor (e.g., computing system 400A having processor 404) receiving a sequence profile for a target patient having an unknown biological state (block 442). The target patient may be distinguishable from the previously discussed reference patients as the biological state of target patient may be unknown and therefore subject to the analysis and determination by applying the trained machine learning model. For example, the application of the trained machine learning model may be determine whether the patient has a disease state or is an AHN patient, whether the patient has a first disease state, whether the patient has a second disease state, etc. Thus, reference patients, the ROIs and sequence profile concerning the reference patients, as well as the biological states of the reference patients may be applicable for the training phase 420, whereas the target patient, as well as the sequence profile and ROIs of the sequence profile, as well as the biological state of the target patient to be determined, may be applicable for the application phase 440.

In some embodiments, the received sequence profile may be unstructured and a processor (e.g., a natural language processor, image processor, a special purpose gene sequencing processor, etc.) may process, translate, decrypt, decipher, and/or quantify the unstructured data into a format that can be vectorized.

At block 444, the computer system may filter the sequence profile based on lncRNA and/or pgRNA. For example, as previously discussed, sequences that do not align (e.g., at least substantially) with sequence identities representing various lncRNA and/or pgRNA may be removed from the sequence profile of the target patient for analysis. The filtering of the sequence profile may strengthen the ability of the machine learning model to accurately determine a classification of a biological state of the target patient by removing aspects of the sequence profile that may trigger unwanted signal noise. Furthermore, by restricting the sequence profile, the machine learning model may run more efficiently towards providing the classification.

At block 446, the computer system may vectorize the received sequence profile of the target patient to generate an input feature vector. For example, the computer system may process unstructured data comprising the sequence profiles to quantify, as feature vectors, relevant information for the filtered sequence profiles. In some embodiments, such relevant information may include one or more values for each of a set of parameters for the filtered sequence profile. In some embodiments, the set of parameters may correspond to a plurality of regions of interest (ROI) of the filtered sequence profile. The values may thus correspond to a level of expression for each of the ROIs. Also or alternatively, the values may correspond to a level of expression of the ROI that is above a predetermined threshold, where the predetermined threshold may be based on a reference expression level for the ROI.

At block 448, the computer system may apply the input feature vector to the trained machine learning model (e.g., as output from block 432) to generate an output feature vector identifying one or more biological states based on the filtered sequence profile. As previously discussed, the trained machine learning model may have a stored set of weights that may indicate the significance of each of a plurality of parameters, such as the presence of ROIs and their respective expression levels, towards a classification of one or more biological state. In at least one embodiment, the output feature vector may be based on a vector of one or more values representing a one or more respective biological states. Thus, in such an embodiment, each value may represent a binary representation (e.g., truth (“1”) or false (“0”)) of a respective biological state (e.g., AHN, first disease state, second disease state, etc.). In some embodiments, the plurality of parameters of the input feature vector may include, comprise, and/or correspond to the parameters represented by the reference input feature vectors used for training the machine learning model. Thus, the input feature vector may be associated with the set of weights in the trained machine learning model to generate the output feature vector determining classification of one or more biological states.

The computer system may then classify the target patient based on the one or more identified biological states (block 450). In some embodiments, such classification may be output as a text, audio, and/or visualization (e.g., via display 412 of computer system 400A). It is contemplated that other machine learning models, training processes, and/or application processes may also or alternatively be implemented for the classification of biological states for the target patient.

### II. Biological and Disease States

Methods and kits discussed herein concern identifying an individual as having a discernable biological state based on the presence, absence, or level of genetic elements. It may be employed with respect to an individual who has tested positive for such disease or biological states, who has one, two, three, four or more symptoms of a condition, disease, or biological state, or who is or has been deemed to be at risk for developing such a disease, biological state, or condition. A “biological state” in the context of the aspects discussed herein refers to a genetic profile with respect to a set of filtered sequences of double-stranded RNA. In some aspects the double-stranded RNA comprises long non-coding RNA (lncRNA) and/or pseudogene RNA (pgRNA) and/or a reference genome. The biological state may refer to more specifically the genetic profile correlated with a particular medical condition or disease or qualification of a particular medical condition or disease. Moreover, the biological state of a single patient may change so it is contemplated that the biological state of an individual may be evaluated more than once. Time will have elapsed between the first and second profiling, and the individual may also have been subjected to other changes such as a treatment or other therapy or other physical changes. For example, a subject may be diagnosed or deemed at risk for a disease or condition. The subject may then be assessed using methods described herein to further assess or continue assessing their biological state classification. In some aspects, the subject may be assessed before and after receiving a treatment and/or at time during the treatment. A subject or individual may be evaluated or be evaluated at least 1, 2, 3, 4, 5, 6, 7, 8, 9, 10, 11, 12, 13, 14, 15, 16, 17, 18, 19, 20 or more times with respect to the same biological state classification(s) or different biological state classification(s).

The disease state may include or exclude a neurological disease. The neurological disease may include or exclude any neurological disease known in the art or, for example, Absence of the Septum Pellucidum, Acid Lipase Disease, Acute Disseminated Encephalomyelitis, Adrenoleukodystrophy, Agenesis of the Corpus Callosum, Agnosia, Aicardi Goutieres Syndrome Disorder, Aicardi Syndrome, Alexander Disease, Alpers Disease, ALS Amyotrophic Lateral Sclerosis, Alternating Hemiplegia, Alzheimer's Disease, Amyotrophic Lateral Sclerosis ALS, Anencephaly, Angelman Syndrome, Antiphospholipid Syndrome, Aphasia, Apraxia, Arachnoid Cysts, Arachnoiditis, Arteriovenous Malformation, Asperger Syndrome, Ataxia Telangiectasia, Ataxias and Cerebellar or Spinocerebellar Degeneration, Atrial Fibrillation and Stroke, Attention Deficit Hyperactivity Disorder, Autism, Autism Spectrum Disorder, Back Pain, Barth Syndrome, Batten Disease, Behcet's Disease, Bell's Palsy, Benign Essential Blepharospasm, Binswanger's Disease, Brachial Plexus Injuries, Brain and Spinal Tumors, Brown Sequard Syndrome, CADASIL, Canavan Disease, Carpal Tunnel Syndrome, Central Cord Syndrome, Central Pain Syndrome, Central Pontine Myelinolysis, Cephalic Disorders, Cerebellar Degeneration, Cerebellar Hypoplasia, Cerebral Aneurysms, Cerebral Arteriosclerosis, Cerebral Atrophy, Cerebral Cavernous Malformation, Cerebral Hypoxia, Cerebral Palsy, Cerebro Oculo Facio Skeletal Syndrome COFS, Charcot Marie Tooth Disease, Chiari Malformation, Chorea, Chronic Inflammatory Demyelinating Polyneuropathy CIDP, Chronic Pain, Coffin Lowry Syndrome, Colpocephaly, Coma, Complex Regional Pain Syndrome, Congenital Myasthenia, Congenital Myopathy, Corticobasal Degeneration, Craniosynostosis, Creutzfeldt Jakob Disease, Cushing's Syndrome, Dandy Walker Syndrome, Deep Brain Stimulation for Parkinson's Disease, Dementia, Dementia With Lewy Bodies, Dermatomyositis, Developmental Dyspraxia, Diabetic Neuropathy, Dravet Syndrome, Dysautonomia, Dysgraphia, Dyslexia, Dyssynergia Cerebellaris Myoclonica, Dystonias, Empty Sella Syndrome, Encephalitis Lethargica, Encephaloceles, Encephalopathy, Epilepsy, Erb Duchenne and Dejerine Klumpke Palsies, Essential Tremor, Fabry Disease, Fahr's Syndrome, Familial Periodic Paralyses, Farber's Disease, Febrile Seizures, Fibromuscular Dysplasia, Foot Drop, Friedreich's Ataxia, Frontotemporal Dementia, Gaucher Disease, Generalized Gangliosidoses, Gerstmann's Syndrome, Gerstmann Straussler Scheinker Disease, Giant Axonal Neuropathy, Glossopharyngeal Neuralgia, Guillain Barré Syndrome, Headache, Hemicrania Continua, Hemifacial Spasm, Hereditary Neuropathies, Hereditary Spastic Paraplegia, Herpes Zoster Oticus, Holmes Adie syndrome, Holoprosencephaly, Huntington's Disease, Hydranencephaly, Hydrocephalus, Hydromyelia, Hypersomnia, Hypertonia, Hypotonia, Inclusion Body Myositis, Incontinentia Pigmenti, Infantile Neuroaxonal Dystrophy, Infantile Refsum Disease, Infantile Spasms, Inflammatory Myopathies, Iniencephaly, Isaac's Syndrome, Joubert Syndrome, Kearns Sayre Syndrome, Kennedy's Disease, Kleine Levin Syndrome, Klippel Feil Syndrome, Klippel Trenaunay Syndrome KTS, Kliiver Bucy Syndrome, Krabbe Disease, Kuru, Lambert Eaton Myasthenic Syndrome, Landau Kleffner Syndrome, Learning Disabilities, Leigh's Disease, Lennox Gastaut Syndrome, Lesch Nyhan Syndrome, Leukodystrophy, Lipid Storage Diseases, Lipoid Proteinosis, Lissencephaly, Locked In Syndrome, Machado Joseph Disease, Megalencephaly, Melkersson Rosenthal Syndrome, Meningitis and Encephalitis, Menkes Disease, Meralgia Paresthetica, Metachromatic Leukodystrophy, Microcephaly, Migraine, Miller Fisher Syndrome, Mitochondrial Myopathies, Mitochondrial Myopathy, Moebius Syndrome, Monomelic Amyotrophy, Motor Neuron Diseases, Moyamoya Disease, Mucolipidoses, Mucopolysaccharidoses, Multi Infarct Dementia, Multifocal Motor Neuropathy, Multiple Sclerosis, Multiple System Atrophy, Multiple System Atrophy with Orthostatic Hypotension, Muscular Dystrophy, Myasthenia Gravis, Myoclonus, Myopathy, Myotonia, Myotonia Congenita, Narcolepsy, Neuroacanthocytosis, Neurodegeneration with Brain Iron Accumulation, Neurofibromatosis, Neuroleptic Malignant Syndrome, Neurological Complications of AIDS, Neurological Complications of Lyme Disease, Neurological Consequences of Cytomegalovirus Infection, Neurological Sequelae Of Lupus, Neuromyelitis Optica, Neuronal Migration Disorders, Neurosarcoidosis, Neurosyphilis, Neurotoxicity, Niemann Pick Disease, Normal Pressure Hydrocephalus, Occipital Neuralgia, Ohtahara Syndrome, Olivopontocerebellar Atrophy, Opsoclonus Myoclonus, Orthostatic Hypotension, Paraneoplastic Syndromes, Paresthesia, Parkinson's Disease, Paroxysmal Choreoathetosis, Paroxysmal Hemicrania, Parry Romberg, Pelizaeus Merzbacher Disease, Peripheral Neuropathy, Periventricular Leukomalacia, Pervasive Developmental Disorders, Pinched Nerve, Piriformis Syndrome, Pituitary Tumors, Polymyositis, Pompe Disease, Porencephaly, Post Polio Syndrome, Postural Tachycardia Syndrome, Primary Lateral Sclerosis, Progressive Multifocal Leukoencephalopathy, Progressive Supranuclear Palsy, Prosopagnosia, Pseudotumor Cerebri, Psychogenic Movement, Rasmussen's Encephalitis, Refsum Disease, Repetitive Motion Disorders, Restless Legs Syndrome, Rett Syndrome, Reye's Syndrome, Sandhoff Disease, Schilder's Disease, Schizencephaly, Septo Optic Dysplasia, Shaken Baby Syndrome, Shingles, Sjagren's Syndrome, Sleep Apnea, Sotos Syndrome, Spasticity, Spina Bifida, Spinal Cord Infarction, Spinal Cord Injury, Spinal Muscular Atrophy, Stiff Person Syndrome, Striatonigral Degeneration, Stroke, Sturge Weber Syndrome, Subacute Sclerosing Panencephalitis, SUNCT Headache, Swallowing Disorders, Sydenham Chorea, Syncope, Syringomyelia, Tabes Dorsalis, Tardive Dyskinesia, Tarlov Cysts, Tay Sachs Disease, Tethered Spinal Cord Syndrome, Thoracic Outlet Syndrome, Thyrotoxic Myopathy, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vasculitis Syndromes of the Central and Peripheral Nervous Systems, Von Hippel Lindau Disease VHL, Wallenberg's Syndrome, Wernicke Korsakoff Syndrome, Whiplash, Whipple's Disease, Williams Syndrome, Wilson Disease, Zellweger Syndrome, Primary Age-Related Tauopathy (PART) dementia (with NFTs similar to AD, but without amyloid plaques), Chronic traumatic encephalopathy (CTE), Vacuolar tauopathy, Lytico-Bodig disease (Parkinson-dementia complex), Ganglioglioma, Gangliocytoma, Meningioangiomatosis, Postencephalitic parkinsonism, Pantothenate Kinase-Associated Neurodegeneration, or Lipofuscinosis.

The disease state may include or exclude cancer. The cancer may include or exclude any cancer known in the art or, for example, epithelial cancer, (e.g., breast, gastrointestinal, lung), prostate cancer, bladder cancer, lung (e.g., small cell lung) cancer, colon cancer, ovarian cancer, brain cancer, gastric cancer, renal cell carcinoma, pancreatic cancer, liver cancer, esophageal cancer, head and neck cancer, or a colorectal cancer. The cancer may include or exclude one or more of the following cancers: adenocortical carcinoma, agnogenic myeloid metaplasia, AIDS-related cancers (e.g., AIDS-related lymphoma), anal cancer, appendix cancer, astrocytoma (e.g., cerebellar and cerebral), basal cell carcinoma, bile duct cancer (e.g., extrahepatic), bladder cancer, bone cancer, (osteosarcoma and malignant fibrous histiocytoma), brain tumor (e.g., glioma, brain stem glioma, cerebellar or cerebral astrocytoma (e.g., pilocytic astrocytoma, diffuse astrocytoma, anaplastic (malignant) astrocytoma), malignant glioma, ependymoma, oligodenglioma, meningioma, meningiosarcoma, craniopharyngioma, haemangioblastomas, medulloblastoma, supratentorial primitive neuroectodermal tumors, visual pathway and hypothalamic glioma, and glioblastoma), breast cancer, bronchial adenomas/carcinoids, carcinoid tumor (e.g., gastrointestinal carcinoid tumor), carcinoma of unknown primary, central nervous system lymphoma, cervical cancer, colon cancer, colorectal cancer, chronic myeloproliferative disorders, endometrial cancer (e.g., uterine cancer), ependymoma, esophageal cancer, Ewing's family of tumors, eye cancer (e.g., intraocular melanoma and retinoblastoma), gallbladder cancer, gastric (stomach) cancer, gastrointestinal carcinoid tumor, gastrointestinal stromal tumor (GIST), germ cell tumor, (e.g., extracranial, extragonadal, ovarian), gestational trophoblastic tumor, head and neck cancer, hepatocellular (liver) cancer (e.g., hepatic carcinoma and heptoma), hypopharyngeal cancer, islet cell carcinoma (endocrine pancreas), laryngeal cancer, laryngeal cancer, leukemia, lip and oral cavity cancer, oral cancer, liver cancer, lung cancer (e.g., small cell lung cancer, non-small cell lung cancer, adenocarcinoma of the lung, and squamous carcinoma of the lung), lymphoid neoplasm (e.g., lymphoma), medulloblastoma, ovarian cancer, mesothelioma, metastatic squamous neck cancer, mouth cancer, multiple endocrine neoplasia syndrome, myelodysplastic syndromes, myelodysplastic/myeloproliferative diseases, nasal cavity and paranasal sinus cancer, nasopharyngeal cancer, neuroblastoma, neuroendocrine cancer, oropharyngeal cancer, ovarian cancer (e.g., ovarian epithelial cancer, ovarian germ cell tumor, ovarian low malignant potential tumor), pancreatic cancer, parathyroid cancer, penile cancer, cancer of the peritoneal, pharyngeal cancer, pheochromocytoma, pineoblastoma and supratentorial primitive neuroectodermal tumors, pituitary tumor, pleuropulmonary blastoma, lymphoma, primary central nervous system lymphoma (microglioma), pulmonary lymphangiomyomatosis, rectal cancer, renal cancer, renal pelvis and ureter cancer (transitional cell cancer), rhabdomyosarcoma, salivary gland cancer, skin cancer (e.g., non-melanoma (e.g., squamous cell carcinoma), melanoma, and Merkel cell carcinoma), small intestine cancer, squamous cell cancer, testicular cancer, throat cancer, thymoma and thymic carcinoma, thyroid cancer, tuberous sclerosis, urethral cancer, vaginal cancer, vulvar cancer, Wilms' tumor, and post-transplant lymphoproliferative disorder (PTLD), abnormal vascular proliferation associated with phakomatoses, edema (such as that associated with brain tumors), or Meigs' syndrome.

The disease state may include or exclude an autoimmune condition. The autoimmune disease may include or exclude one or more of diabetes, graft rejection, GVHC, arthritis (rheumatoid arthritis such as acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis, x-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica (NMO), inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), bowel inflammation, pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, proliferative nephritis, autoimmune polyglandular endocrine failure, balanitis including balanitis circumscripta plasmacellularis, balanoposthitis, erythema annulare centrifugum, erythema dyschromicum perstans, eythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema including allergic or atopic eczema, asteatotic eczema, dyshidrotic eczema, and vesicular palmoplantar eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, systemic lupus erythematosus (SLE) such as cutaneous SLE or subacute cutaneous SLE, neonatal lupus syndrome (NLE), and lupus erythematosus disseminatus, juvenile onset (Type I) diabetes mellitus, including pediatric insulin-dependent diabetes mellitus (IDDM), and adult onset diabetes mellitus (Type II diabetes) and autoimmune diabetes. Also contemplated are immune responses associated with acute and delayed hypersensitivity mediated by cytokines and T-lymphocytes, sarcoidosis, granulomatosis including lymphomatoid granulomatosis, Wegener's granulomatosis, agranulocytosis, vasculitides, including vasculitis, large-vessel vasculitis (including polymyalgia rheumatica and gianT cell (Takayasu's) arteritis), medium-vessel vasculitis (including Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis, such as Churg-Strauss vasculitis or syndrome (CSS) and ANCA-associated small-vessel vasculitis, temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), Addison's disease, autoimmune neutropenia, pancytopenia, leukopenia, diseases involving leukocyte diapedesis, CNS inflammatory disorders, Alzheimer's disease, Parkinson's disease, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Behcet's disease/syndrome, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, thermal injury, preeclampsia, an immune complex disorder such as immune complex nephritis, antibody-mediated nephritis, polyneuropathies, chronic neuropathy such as IgM polyneuropathies or IgM-mediated neuropathy, autoimmune or immune-mediated thrombocytopenia such as idiopathic thrombocytopenic purpura (ITP) including chronic or acute ITP, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), experimental autoimmune encephalomyelitis, myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, gianT cell hepatitis, chronic active hepatitis or autoimmune chronic active hepatitis, lymphoid interstitial pneumonitis (LIP), bronchiolitis obliterans (non-transplant) vs NSIP, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal or segmental or focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia greata, alopecia totalis, CREST syndrome (calcinosis, Raynaud's phenomenon, esophageal dysmotility, sclerodactyl), and telangiectasia), male and female autoimmune infertility, e.g., due to anti-spermatozoan antibodies, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, parasitic diseases such as leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, SCID, acquired immune deficiency syndrome (AIDS), echovirus infection, sepsis, endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, gianT cell polymyalgia, chronic hypersensitivity pneumonitis, keratoconjunctivitis sicca, epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders, asperniogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, non-malignant thymoma, vitiligo, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, autoimmune polyglandular syndrome type I, adult-onset idiopathic hypoparathyroidism (AOIH), cardiomyopathy such as dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, and disease accompanying vascularization, allergic hypersensitivity disorders, glomerulonephritides, reperfusion injury, ischemic re-perfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, graft versus host disease, contact hypersensitivity, asthmatic airway hyperreaction, and endometriosis.

The disease state may include or exclude a blood clotting disorder. The blood clotting disorder may include or exclude Von Willebrand disease, Haemophilia A, Haemophilia B, Haemophilia C, Factor V deficiency, Factor X deficiency, Factor VII deficiency, Factor XIII deficiency, Prothrombin deficiency, afibrinogenemia, hereditary thrombophilia, antithrombin III deficiency, protein C deficiency, protein S deficiency, Factor V Leiden, prothrombin mutation (gene 20210A mutation), antiphospholipid antibody syndrome, increased levels of factors VIII, IX, XI, or fibrinogen, or fibrinolysis defect.

The disease state may include or exclude cardiovascular disease. Cardiovascular disease may include or exclude coronary heart disease, stroke, peripheral arterial disease, aortic disease, atherosclerosis, heart attack, angina pectoris, arrhythmia, dysrhythmia, cardiac ischemia, high cholesterol, heart failure, high blood pressure, venous thromboembolism, aortic aneurysm, cerebrovascular disease, rheumatic heart disease, congenital heart disease, deep vein thrombosis, and pulmonary embolism.

The disease state may include or exclude a post-viral syndrome. The post-viral syndrome may include or exclude long covid, myalgic encephalomyelitis (ME), chronic fatigue syndrome (CFS), myalgia, fibromyalgia, virus-induced myocarditis, Gulf War illness, and post-polio syndrome.

### III. Kits

Certain aspects of the present invention also concern kits containing compositions of the invention or compositions to implement methods of the invention. In some embodiments, kits can be used to evaluate one or more biomarkers. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules, antibodies, or inhibitors, or any value or range and combination derivable therein. In some embodiments, there are kits for evaluating biomarker activity or level in a single cell.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, antibodies, synthetic nucleic acids, nonsynthetic nucleic acids, and/or inhibitors of the disclosure for prognostic or diagnostic applications are included as part of the disclosure. Specifically contemplated are any such molecules corresponding to any biomarker identified herein, which includes antibodies that bind to such biomarkers as well as nucleic acid primers/primer sets and probes that are identical to or complementary to all or part of a biomarker, which may include noncoding sequences of the biomarker, as well as coding sequences of the biomarker.

In certain aspects, negative and/or positive control nucleic acids, antibodies, probes, and inhibitors are included in some kit embodiments. In addition, a kit may include a sample that is a negative or positive control for methylation of one or more biomarkers.

It is contemplated that any method or composition described herein can be implemented with respect to any other method or composition described herein and that different embodiments may be combined. The claims originally filed are contemplated to cover claims that are multiply dependent on any filed claim or combination of filed claims.

### IV. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Classification of Biological and Disease States Using Different Machine Learning Algorithms

It was found that biological states, such as disease and non-disease and different disease can be determined by filtering sequencing data obtained from subject on long non-coding RNAs (lncRNA) or pseudogene RNA (pgRNA).

Blood samples were collected in PAXgene tubes (Qiagen, Germany) from patients diagnosed with Relapsing-Remitting Multiple Sclerosis, Mild Cognitive Impairment/Alzheimer's Disease (MCI/AD), Parkinson's Disease, Amyotrophic Lateral Sclerosis, or apparently healthy controls (Normals). Frozen blood is thawed from −80° C. to room temperature, inverted several times to resuspend pelleted blood components and kept at room temperature for 4 hours to ensure completion of red blood cell lysis. Blood is then stored at +4° C. overnight. Prior to RNA extraction, blood is brought to room temperature. RNA extraction is carried out using PAXgene Blood RNA Kit v2 (PreAnalytix) according to manufacturer instructions with the following modification: final elution is carried out with 25 μL of 10 mM Tris to increase final concentration for subsequent steps. To check the RNA quality, 1 μL of each RNA sample is checked on a Bioanalyzer 2100 (Agilent) using RNA Nano Chips (Agilent SKU #5067-1511) according to manufacturer instructions. Following confirmation of high quality RNA, the samples are transferred to PCR tubes and volume is brought up to 26 μL. Tubes are then transferred to the thermal cycler and ran with the following program: 1) 70° C.—1 minute, 2) 40° C.—2 minutes. Samples are removed from the thermal cycler. Next, 3 μL of RNAse R buffer and 1 μL of RNAse R enzyme are added to each sample and mixed completely. The samples are then centrifuged and transferred to the thermal cycler and incubated at 40° C. for 120 minutes. Following RNAse R digestion, RNAse R resistant RNA is precipitated. 2 μL of Glycogen (20 mg/mL) (Thermo Scientific SKU #R0561) is added to each sample. Next, 3 μL of 3M Sodium Acetate (Ambion SKU #AM9740) is added to each sample, and samples are mixed. Then, 90 μL of 100% Ethanol is added to each sample and mixed completely. The samples are then stored at −20° C. for at least 8 hours. Samples are placed in a pre-chilled refrigerated centrifuge and spun at maximum speed for 10 minutes. The supernatant is removed and 200 μL of room temperature 80% Ethanol is added. Samples are then centrifuged at maximum speed for 1 minute, followed by supernatant removal. Samples are then spun for 5 seconds, and any remaining supernatant is removed. Samples are air dried and resuspended in 8.5 μL RNAse free 10 mM Tris. To check the quality of the RNA, 1 μL of each RNA sample is checked on a Bioanalyzer 2100 (Agilent) using RNA Nano Chips (Agilent SKU #5067-1511) according to manufacture instructions. Next, RNA-seq libraries are made using YourSeq v1.6 in Full Transcript mode (Amaryllis Nucleics) according to manufacturer instructions with the following modification: RNA fragmentation step is reduced to 60 seconds at 94° C. Samples are washed with 2× bead/sample ratio using included Carboxyl size selection beads. All samples are run on a Bioanalyzer 2100 (Agilent) using Hi Sensitivity DNA chips (Agilent SKU #5067-4626). Concentrations for pooling were determined between the intervals of 250 bp and 600 bp. Volumes for equimolar amounts of each sample were determined based on interval concentrations and duplicate pools were made. Size selection on the final pools were done using a Pippin HT (Sage Science). Libraries were size selected for fragments between 250 bp and 600 bp. Final pools were combined and concentrated using Ampure XP Beads (Beckman Coulter) at a 2× bead/sample ratio. Bead pellet was washed 2× with 80% Ethanol and allowed to air dry. Beads were resuspended in 40 μL of 10 mM Tris and transferred to fresh tube from magnetic plate to exclude transfer of beads. All samples are run on a Bioanalyzer 2100 (Agilent) using Hi Sensitivity DNA chips (Agilent SKU #5067-4626). The final pool is run in duplicate at a dilution of ¼ concentration and in duplicate at full concentration. The library pools are then sequenced to determined biomarker levels.

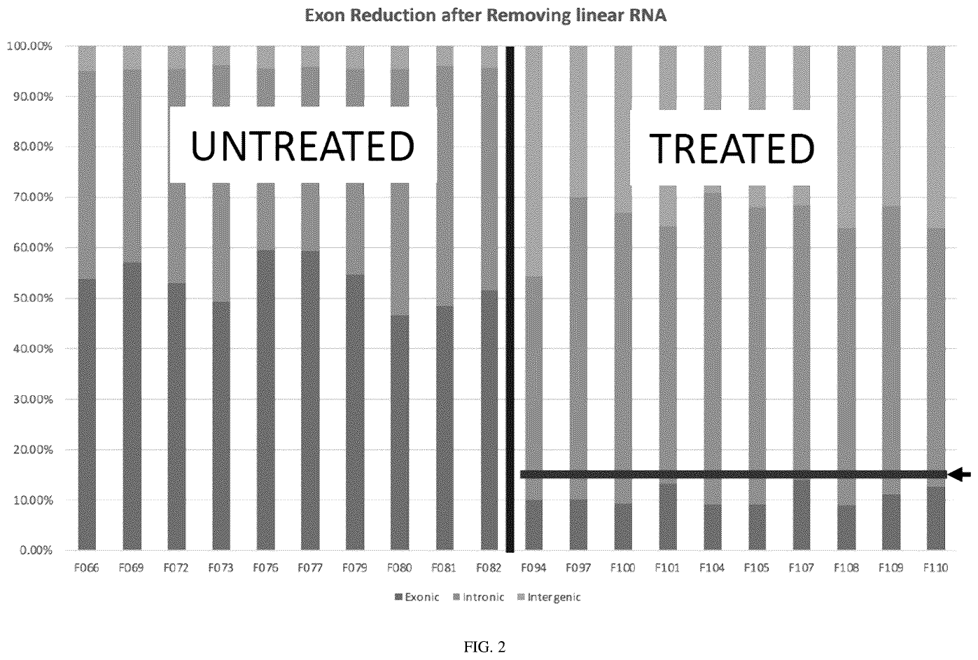

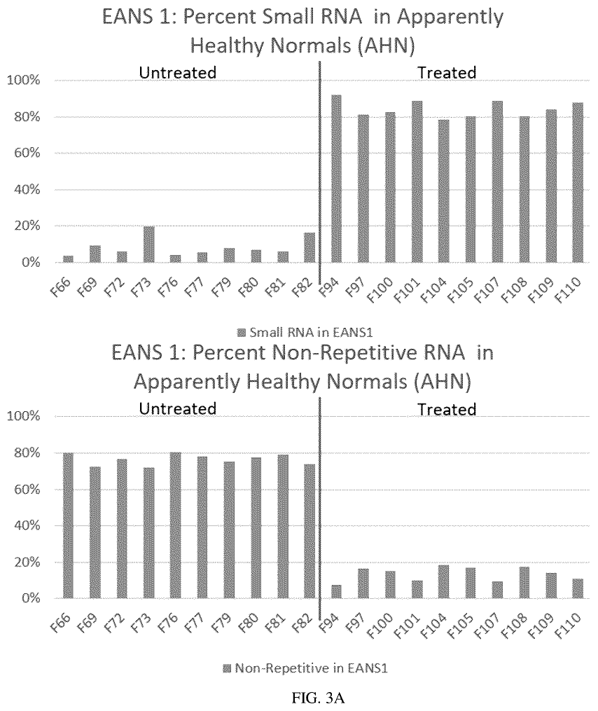

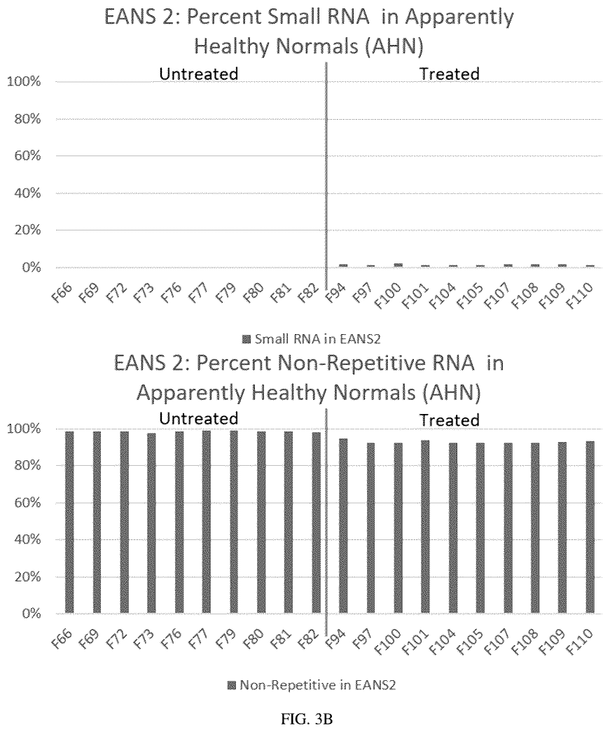

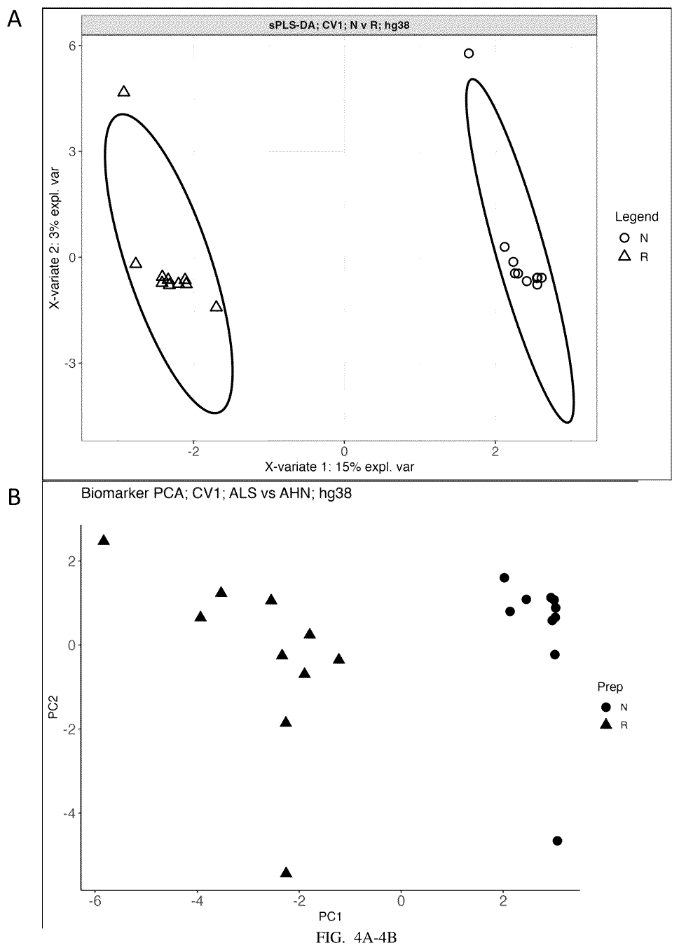

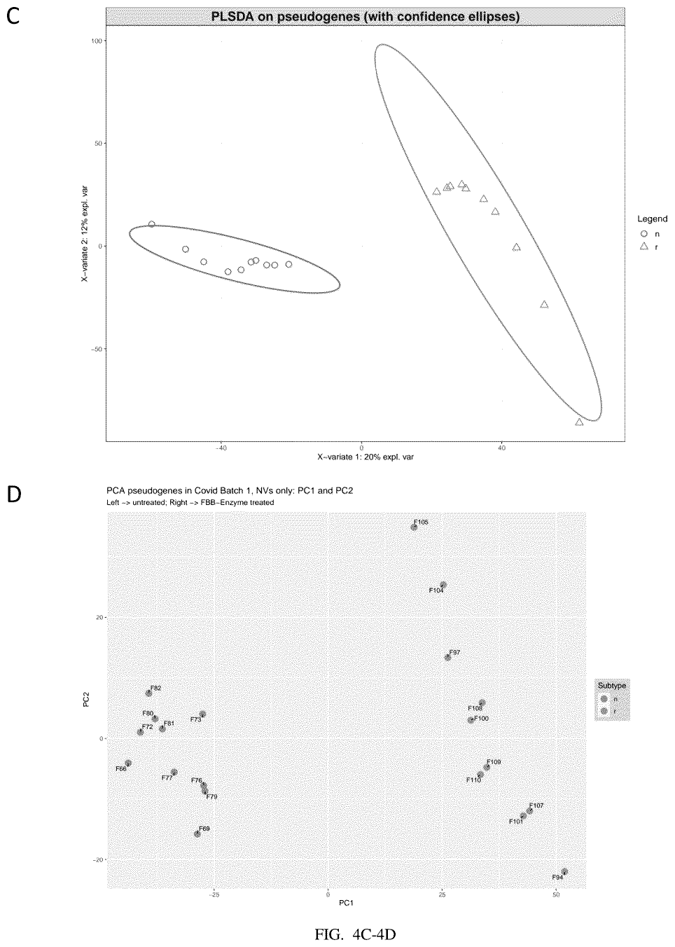

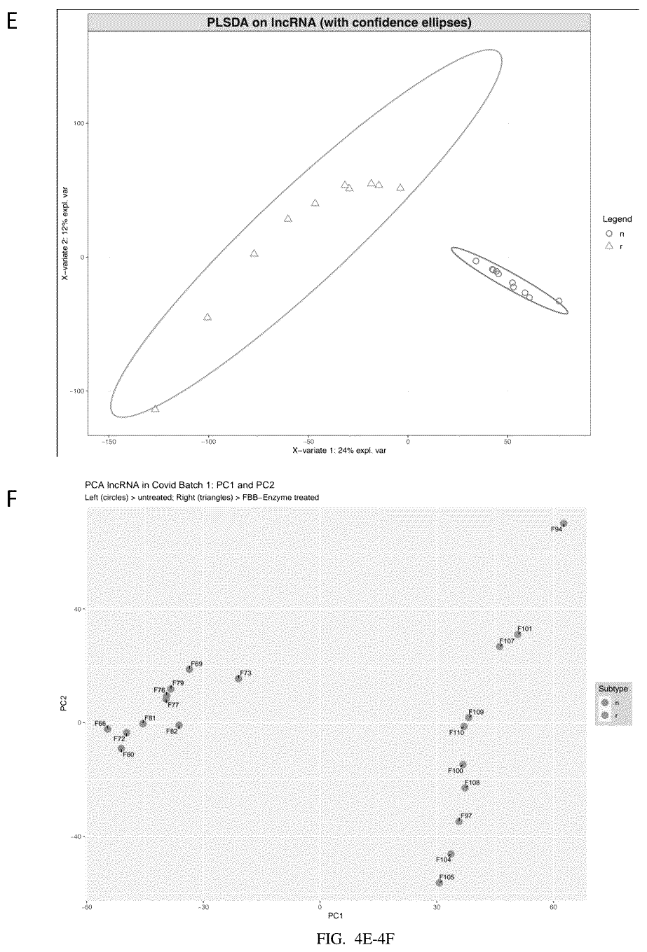

To test for efficient processing of the samples, exonic RNA, GC content, small RNA percentage, and non-repetitive RNA percentage. As shown in FIG. 2, treating the samples with RNAse R to deplete linear RNA reduces the exonic content of the sample to less than 14%. RNAse R treated samples were also found to have a significantly greater percentage of small RNA and a significantly reduced amount of non-repetitive RNA, compared to untreated, in lncRNA-filtered data (FIG. 3A). However, this difference was not seen in pgRNA filtered data (FIG. 3B). As shown in FIG. 4, different Bioinformatics/Machine Learning Tools can measure clusters in RNA samples depleted of linear RNA. It was also found that different Bioinformatics/Machine Learning Tools can measure clusters in RNA samples that were not depleted of linear RNA.

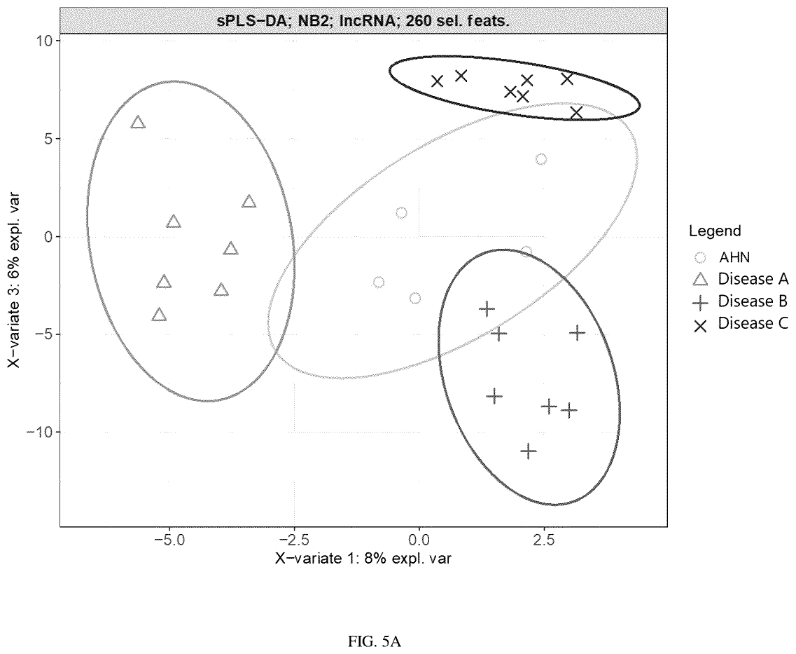

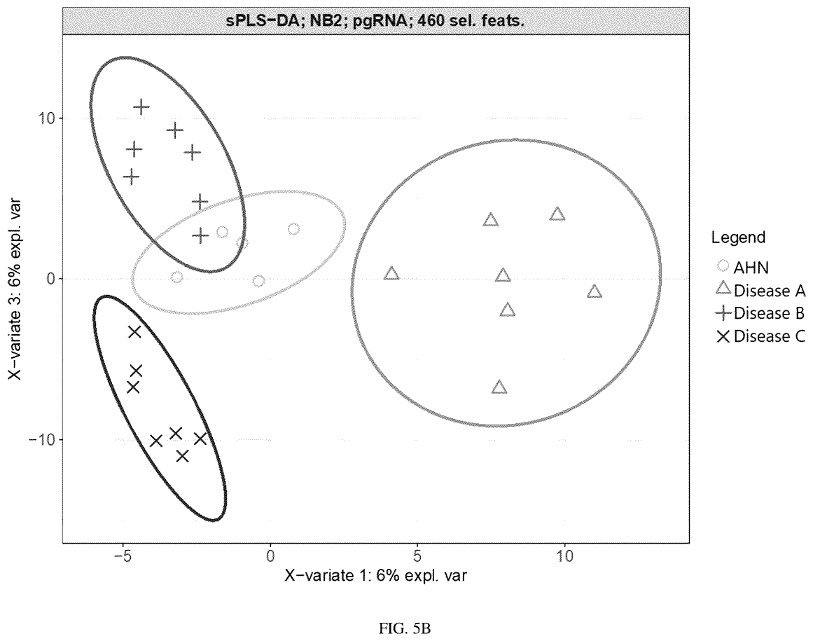

Samples from healthy individuals (AHN—apparently healthy normal) and from individuals with various diseases were processed as described above. The sequence data was filtered using either a EANS (Endogenous Ancestral Nucleotide Sequences) 1 database (long non-coding RNA (lncRNA)) or EANS 2 database (pseudogenes (pgRNA)). As shown in FIGS. 5A-5B, the methods were able to group healthy subjects separately from subjects having various different diseases. The methods described herein were able to distinguish apparently healthy normal subjects from subjects having various neurological diseases, such as multiple cognitive impairment, multiple sclerosis, Parkinson's disease, and amyotrophic lateral sclerosis. The methods were also capable of separating apparently healthy normal subjects from those having long covid, psoriatic arthritis, multiple system atrophy, and various cancers, such as breast, colon, head and neck, non-small cell lung cancer, pancreatic cancer, prostate cancer, and brain cancer. The provided data demonstrates that either eansRNA-enriched sequences can be used with various machine learning algorithms to group individuals of different diseases.

### Example 2: Endogenous Ancestral Nucleotide Sequences in Therapeutic Drug Monitoring of Immunosuppressive Drugs

A recent review highlighted the lack of biomarkers for monitoring immunosuppressive drugs (ISD).¹ The reviewed biomarkers for therapeutic drug monitoring (TDM) of ISDs predominantly measure the outcomes of pathological occurrences such as organ failure and the effects of a disease, rather than delving into their fundamental etiopathological mechanisms, the cause of disease. With this background, it was found that endogenous ancestral nucleic sequences (EANS) play a role in diseases in both humans and non-humans.² There is an abundance of ignored genetic elements as diagnostic and therapeutic targets. They encompass a vast array of categories, including endogenous viruses, retrotransposons, transfer RNA (tRNA), ribosomal RNA (rRNA), small nuclear RNA (snRNA), small nucleolar RNA (snoRNA), microRNAs (miRNAs), long non-coding RNA (lncRNAs), pseudogenes, piwi-interacting RNA (piRNAs), and ribonuclease P RNA. Consequently, the evaluation of EANS, particularly eansRNA, in contrast to eansDNA, presents potential clinical importance by enabling the monitoring of intricate multifaceted RNA ecosystems, thus facilitating comprehension of the fundamental causes and controls of many diseases.

For years, the challenge has always been to measure the expression of clinically relevant eansRNAs.³ Sequences from RNAseq data were taken and organized into the various categories of RNA listed above. With machine learning, two dominant approaches emerged for understanding the large dynamic changes in eansRNA variance, either through principal component analysis (PCA) or bundled heat maps.

Therefore, two models were developed using the selected machine learning formats. The first model studies the therapeutic use of monitoring eansRNAs over time, especially after ISD therapy. The second model provides useful insight into the dynamics of emergence and reemergence of eansRNA ecosystems over time from the blood collection of patients with Mild Cognitive Impairment (MCI).

### A. Introduction

EANS are panbiotic non-coding nucleotide sequences that exhibit homology with both human and non-human genomic sequences, as identified through the Basic Local Alignment Search Tool (BLAST), a bioinformatics web application for comparing a query sequence against a database of sequences to find regions of similarity.⁴ This shared genetic similarity across diverse species underscores the evolutionary connections between different organisms and points to the ancient microbial origins of many eansRNA sequences. Horizontal gene transfer and endogenous viral element integration are considered significant contributors to the evolution of EansRNA repertoires in humans.⁵ EansRNA are also often found in genomic regions rich in transposable elements, endogenous retroviruses, and other repetitive sequences, which are known to originate from microbial integration over evolutionary time. Accumulating evidence suggests that dysregulation of eansRNAs is involved in the initiation and progression of human diseases such as cancer, neurodegeneration, and autoimmune and cardiovascular diseases. For example, in some cancers, eansRNAs can act as oncogenes or tumor suppressors, regulating cellular processes such as cell proliferation, apoptosis, metastasis, and angiogenesis. The lncRNA HOTAIR (HOX transcript antisense RNA) is upregulated in various cancers and promotes cancer progression by inducing genome-wide re-targeting of the polycomb repressive complex 2 (PRC2), leading to altered histone methylation patterns and gene expression changes. Conversely, lncRNA GAS5 (Growth Arrest-Specific 5) functions as a tumor suppressor by regulating apoptosis and cell cycle arrest.⁶

In neurological disorders, eansRNAs have been found to play a role in neurodevelopment, synaptic plasticity, and neuronal survival. For example, lncRNA BACE1-AS is known to be involved in the progression of amyloid deposition in neurodegenerative diseases, as it is frequently upregulated in the brains of patients with Alzheimer's disease (AD).⁷ Dysregulation of eansRNA has also been observed in other neurodegenerative disorders, such as Parkinson's disease (PD).⁸ The pathogenesis of autoimmune diseases has also been linked to dysregulation of eansRNA, such as in Multiple Sclerosis (MS), where studies have shown several eansRNAs (e.g., MALAT and NEAT1) linked to altered immune responses and changes in myelination dynamics.^{9, 10}

EANS-derived RNA, or eansRNA, draws from very different non-coding RNA categories, as described above, and underscores the significance of eansRNAs as essential regulators of health and disease processes.^{2, 11} A systematic approach to quantify all clinically relevant ncRNA species present in a blood sample was developed.

Two models are used to assess the eansRNA ecosystem in patients undergoing ISD therapy. The first model addressed therapeutic application for monitoring patients undergoing ISD therapy. In the first model, post-ISD administration can be effectively tracked by analyzing RNA extracted from blood samples using next-generation sequencing and machine learning (ML) algorithms. This approach enables a comprehensive evaluation of eansRNA expression patterns, facilitating monitoring of disease progression. The eansRNA panels were organized according to eansRNA subcategories, and then an eansRNA panel with ancestral genes with steroid response elements (SREs) that could significantly change the RNA ecosystem in response to ISD was selected. SREs are specific DNA sequences that are involved in the regulatory effects of steroid hormones, including glucocorticoids and mineralocorticoids.

The second model for measuring eansRNA over time focuses on patients with Mild Cognitive Impairment (MCI) undergoing therapeutic plasma exchange (TPE) to treat neurological conditions, allowing for the observation of clusters of eansRNA re-expression dynamics over time following complete plasma exchange. Each eansRNA species can be monitored for re-expression kinetics using the host as the “incubator” for eansRNA expression.

TPE is the process of removing a patient's whole blood via an intravenous line and centrifuging the whole blood to separate cells from the plasma. Once separated from the plasma, red blood cells are mixed with albumin and reinfused into the patient via a second intravenous line. The patient's plasma is discarded, along with cellular detritus, chemical signals, and the landscape of coding and non-coding RNAs packaged in plasma components, such as exosomes. A 2020 trial found that a series of plasma exchanges over a twelve-month period resulted in an overall improvement in symptoms associated with Alzheimer's disease.¹² While this trial showed that TPE is an effective treatment for Alzheimer's disease, questions remain as to why TPE is effective in reducing symptoms in Alzheimer's disease. It is hypothesized that eansRNAs would also be removed by the plasma exchange process and that, as a result of their removal, patients would experience amelioration of their disease state.

A laboratory-developed test was developed for the two models to monitor the eansRNA ecosystem at a sequence-by-sequence level, assessing the expression of each distinct endogenous ancestral gene. These results serve as a conceptual illustration supporting the proposition that the assessment of ancestral genes has significant clinical value in the field of personalized and precision ISD surveillance.

### B. Materials and Methods

1. TPE Blood eansRNA Isolation

Each participant was scheduled to undergo a standard-of-care TPE. On the day of the procedure, each participant underwent a safety screen performed by a nurse performing the procedure. This was performed to ensure that there were no contraindications for completing TPE that day. The TPE Nurse obtained bilateral peripheral IV access using a 17G apheresis needle and an 18G IV angiocath needle in the opposing arm. Prior to the start of the procedure, the nurse collected the two PaxGene tubes. These tubes were labeled pre-procedure and frozen in a −80° C. freezer immediately after collection. Plasma exchange was performed as usual, and at the end of the procedure, the nurse collected two additional PaxGene tubes. These samples were labeled post-procedure and frozen at −80° C. The samples were then sent on dry ice to the FBB-Biomed laboratories for processing.

Patients returned to the clinic seven, fourteen, twenty-one and twenty-eight days post-plasma exchange for follow-up blood collection. Each participant had two PaxGene tubes drawn, labeled, and frozen at −80° C. The samples were then sent to the FBB Biomed Laboratory for further analyses. During the 28 days between TPE and the final blood draw, the participants were not allowed to undergo any additional TPE procedures to prevent confounding of the laboratory results. A specialized and patented FBB Biomed-proprietary RNA enrichment enzymatic technique was used to extract whole-blood RNA, which significantly enhanced the concentration of panbiotic sequences.

2. MCI Blood eansRNA Enrichment

Freshly collected whole blood samples (PAXgene™ tube) were obtained from the MaxWell Clinic from a cohort of three patients diagnosed with Mild Cognitive Impairment (MCI). Following thawing, total RNA was extracted from the whole blood samples using the Qiagen extraction method. A specialized FBB Biomed-proprietary RNA enrichment enzymatic technique was then used to extract whole-blood RNA, significantly enhancing the concentration of panbiotic sequences.

### 3. Sequencing and Bioinformatics

The enriched RNA after extraction and FBB Biomed-proprietary enzymatic treatment was subsequently processed into a comprehensive RNA transcript library and subjected to sequencing using the Illumina™ platform, specifically the NovaSeq 6000 model, employing paired-end sequencing with a read length of either 150 or 80 nucleotides. Sequencing was then performed. The resulting sequences were aligned against FBB Biomed's eansRNA reference databases (representing a proprietary subset derived from the total GRCh38 transcriptome assembly) with the STAR splice-aware aligner, and quantified with salmon “quant.” ¹³,¹⁴ To generate time-course heatmaps, gene-length-scaled read counts were imported into R, and a standard Z-score was applied per feature (row) for normalization. Z-scored count data were plotted using ‘pheatmap’ with hierarchical clustering on rows.

TPE time-course Venn diagrams were generated by filtering RNA count data after data transformation. Briefly, count data were TPM (‘transcripts per million’) normalized to account for differences in library size, a small pseudo-count was added (to prevent taking the log of zero), and the log (base 2) was taken. The difference in logs was calculated to yield a log 2 fold change (log 2FC) between time points. Thresholds were applied to calculate the six RNA response categories. 1) Initial TPE-negative responders: log 2FC<=−2 between post- and pre-TPE. 2) Initial TPE-positive responders: log 2FC>2 between post-TPE and pre-TPE. 3) Recovered negative responders: eansRNA in (1), those who regained >80% of pre-TPE baseline by day 28. 4) Recovered positive responders: of eansRNA in (2), those that decreased to within 1 tpm of their pre-TPE baseline on Day 28. 5) Of the eansRNAs in (1), those that did not recover to within 80% of the pre-TPE baseline. 6) eansRNA was not detected pre-TPE or immediately post-TPE but was present on day 28. Principal component analysis was carried out in R using the ‘prcomp’ function on the log of eansRNA expression counts, with scaling and centering enabled.

Individual participants were required to have an existing diagnosis of Alzheimer's Disease or MCI. Informed consent was obtained from each participant, followed by clinician evaluation to ensure that they met the inclusion criteria and were safe participants to undergo TPE.

### C. Results

Data from a single patient with relapsing-remitting multiple sclerosis (RRMS) who received an immunosuppressive regimen during a three-year surveillance period was analyzed. Using heat maps to visualize eansRNA dynamics over time, significant therapeutic intervention-related changes were observed. Each heatmap row represents an eansRNA sequence and each heatmap column represents a specific blood collection time point. Rows were clustered hierarchically using similar expression dynamics across samples from each visit.

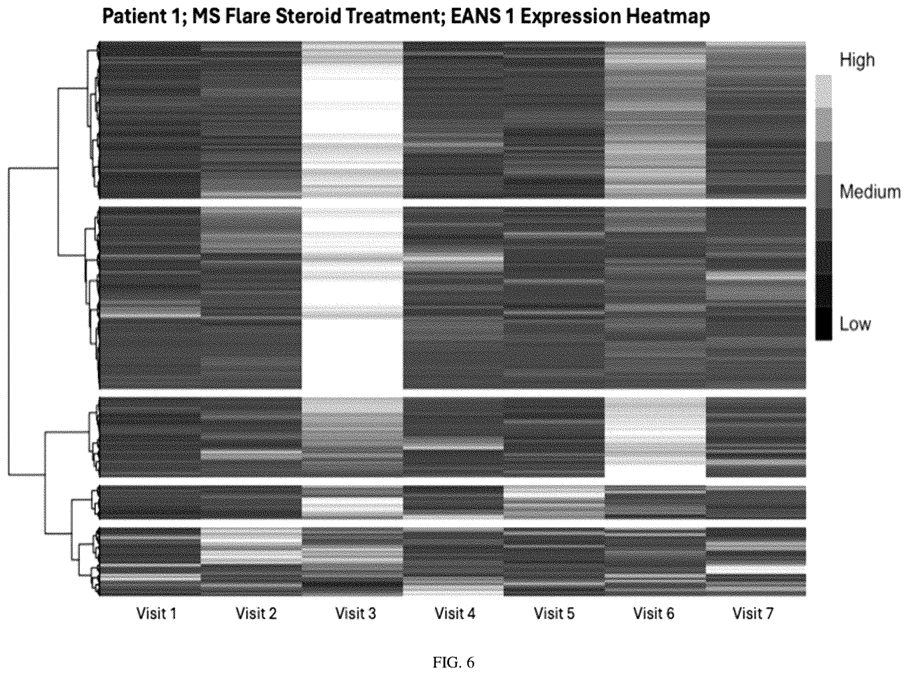

Initially, a marked escalation in eansRNA levels was documented (FIG. 6), aligning temporally with the initiation of immunosuppressive drug treatment weeks prior. After this surge, a major decrease in eansRNA expression was noted six months after subsequent evaluation. Notably, one year after the initial observation, the patient underwent treatment with Prozac (visit six) for a flare-up, coinciding with the reactivation of numerous eansRNA sequences six months post-treatment. These observations underscore the responsive and dynamic nature of eansRNA ecosystems, suggesting their association with ISD monitoring.

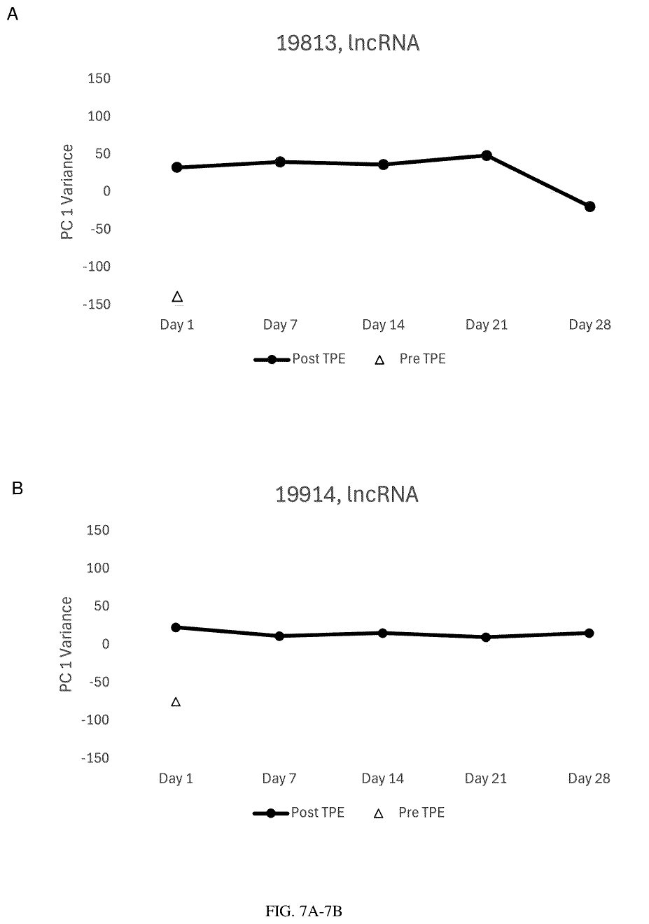

Visible alterations are observed in the PCA (FIG. 7) and visual cluster heat map analyses (data not shown). Another important observation occurred on day 28 in patient 19813 (FIG. 7A), when both PCA and heat maps indicated a shift in the eansRNA ecosystems. There was a noticeable change at day 28, following a consistently stable principal component 1 (PC1) variance over three weeks. The clustered heat map revealed the reappearance of eansRNAs observed prior to TPE initiation, along with the emergence of new eansRNA clusters that were not present during the pre-TPE phase.

With respect to the second model, an examination was conducted to monitor the reappearance of specific eansRNA sequences by quantifying eansRNA expression before the commencement of TPE, at weekly intervals throughout the treatment period, and upon culmination of the study. Three patients with MCI underwent TPE therapy. FIG. 7A illustrates the time-dependent change in the principal component 1 (PC1) variance following principal component analysis (PCA) for one patient (patient 2). A dramatic shift in the profile was noted two hours after the initiation of plasma exchange. Subsequently, there was a period of relative stability in PC1 variance over the ensuing 21 d, punctuated by the emergence and subsequent disappearance of diverse EANS clusters. Noteworthy alterations in PC1 variance were observed on the 28th day, characterized by a significant decline spanning multiple 50-unit intervals along PC1. Intriguingly, a novel cohort of ancestral genes was overexpressed at this juncture, whereas the initial set of overexpressed genes persisted with consistently low counts. This depiction serves as a basis for an EANS load test, tracking all clinically relevant eansRNAs with applications in early stage screening, differential diagnosis, and therapeutic monitoring.

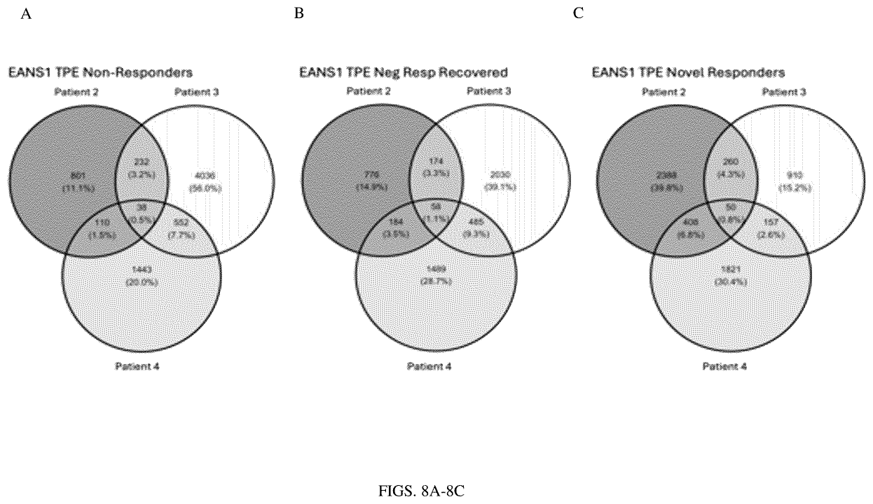

In FIGS. 8A-8C, Venn diagrams are used to visually depict the consistency and variability of specific eansRNA sequences. The TPE approach served as a model for tracking all clinically useful RNAs. Given the strong ISD response of many eansRNAs, some eansRNAs are part of several different hormone-responsive systems, such as the immune system. The facets of the immune system are influenced by putative genomic variable regions obtained as a consequence of evolutionary adaptations owing to human migration.^{16, 17} Such further work, will pave the way for the realization of genuinely personalized precision medicine.

In FIG. 8A-8C, Venn diagrams delineate three distinct populations of eansRNA (as defined in Methods), so-called “TPE non-responders, TPE negative responders/recovered,” and “TPE novel responders,” demonstrating a collective association with three patients undergoing therapeutic plasma exchange (TPE). Specifically, following the TPE procedure, 7212, 5196, and 5994 eansRNA sequences were identified for patients 2, 3, and 4, respectively. While most of the specific eansRNA sequences were unique to each individual, a subset of sequences exhibited similarities with one or both patients with mild cognitive impairment (MCI).

As shown in FIG. 8A, 38 eansRNA sequences were common among all three patients with MCI who did not respond to TPE therapy (i.e., were not re-expressed after being knocked down by TPE).

In FIG. 8B, the subsequent population of eansRNAs comprises those initially eliminated by TPE but displaying re-expression by day 28. While most specific eansRNA sequences remained unique to each patient, a subset of sequences displayed similarities with one or both patients with MCI. Notably, 58 eansRNA sequences were common across all three patients with MCI.

In FIG. 8C, the third population of eansRNAs consists of sequences that exhibited minimal expression during the pre-TPE blood draw but demonstrated expression by day 28. Similar to previous populations, the majority of specific eansRNA sequences were unique to each patient, with a subset showing similarities to MCI patients. Notably, 50 eansRNA sequences were common across all three patients with MCI.

### D. Discussion

Notably, within the broader scope of medical diagnostics, emphasis has traditionally been placed on biomarkers indicative of disease manifestations, that is, the effect of the disease. However, a paradigm shift is currently underway, propelled by advancements in powerful bioinformatics, statistical, and machine learning technologies facilitated by affordable computing capabilities. This transformative juncture provides a unique opportunity to delve deeper into the fundamental origins of diseases, enabling a more comprehensive understanding of their etiology, precise measurements, and targeted interventions specifically tailored to address disruptions within the RNA ecosystem, that is, the cause of disease.

It was found that the onset of disease initiation and progression can be traced back to substantial perturbations within the eansRNA ecosystem. To ascertain and quantify these alterations, a bioinformatic protocol for the surveillance of eansRNA occurrences in the host RNA ecosystem at the time of blood collection was developed. Disturbances in the eansRNA ecosystem result in a series of intricate molecular and cellular events that culminate in disease-specific consequences. Such deviations in the eansRNA ecosystem, illustrated through heat maps, can be monitored using machine learning methodologies to assist in predicting disease onset and recurrence.

Two sophisticated models were specifically developed to monitor and analyze the behavioral characteristics of individual eansRNA sequence categories. The first model focuses on examining the expression levels of each unique RNA in response to a defined ISD treatment regimen. The previous work featured a detailed investigation of an individual with RRMS who was regularly monitored over a three-year period, with particular emphasis on the effects of intravenous ISD administration. A significant alteration in the eansRNA ecosystem just weeks after ISD treatment, as evidenced by Principal Component Analysis (PCA) was revealed. Indeed, the same patient was reevaluated using heat maps for visualization, highlighting their response to ISD. Observation of the recovery profiles of ISD-sensitive eansRNA species post-treatment can provide valuable insights into the therapeutic response dynamics.

Furthermore, use of TPE as a comprehensive framework for investigating the intricate re-expression dynamics involved in replenishing a diverse array of eansRNAs is reasonable. By meticulously analyzing data from a cohort of three MCI patients who underwent TPE, the recovery trajectories of individual eansRNA over a 28-day observation period were systematically documented. These findings facilitate the classification of these trajectories into distinct clinically relevant categories, shedding light on identifying newly constitutively expressed eansRNAs and the resurgence of eansRNA species during the TPE recovery process. This underscores the potential of TPE as a valuable tool for studying RNA dynamics and therapeutic responses in patients with Mild Cognitive Impairment.

The AMBAR Trial, published in 2020, demonstrated that plasma exchange conducted with albumin was more effective than placebo in reversing many of the signs and symptoms of Alzheimer's disease.” However, the authors did not address the duration of symptom improvement between plasma exchanges.¹² Measuring the EANS in patients with Alzheimer's disease and MCI provide clinicians with a tool to personalize the frequency and efficacy of plasma exchange per patient. Testing for EANS, both pre- and post-plasma exchange, allow clinicians to understand how plasma exchange changes a patient's blood chemistry, which in turn drives the expression of EANS and its impact on symptoms.

To fully grasp the personalized nature of this methodology and its implications for ISD monitoring, it is crucial to revisit a seminal study that explores the concept of a human blood DNA virome.¹⁸ This significant work represents a research endeavor involving the examination of blood DNA samples collected from a cohort of 8,000 individuals. Through this investigation, the complex virome composition present in human populations was revealed, laying a solid groundwork for the identification of viral-like sequences in human blood samples.

It is believed that endogenous viruses and other ancestral microbial genes have played a significant role in shaping the genetic diversity of human populations over the last 100,000 years.¹⁶ As such, these ancient viral sequences have contributed to the unique genetic makeup of different ethnic groups and influenced the evolution of human populations as they migrated geographically.

As a result, ancestral microbial genes play a significant role beyond merely providing genetic variability and exerting a substantial influence on shaping human genetic diversity. This then supports the existence of variable and dynamic genomic segments within the human genome, which contribute to the unique traits observed across diverse human populations, thereby offering potential insights for personalized and precise medical interventions. The investigation was initiated based on a publication that recognizes the presence of unmapped regions within large portions of the human genome.²⁰ It is believed that these unmapped regions represent a key focus for exploring genomic variable region hotspots for eansRNA expression.

Given these fundamental principles, tests, such as an EANS load test, that can assess the overall presence and impact of all of these ancestral sequences within the human genome will be developed. Such a test could provide valuable insights into the eansRNA ecosystem and improve the understanding of how these endogenous viruses have influenced human evolution and population diversity.

It is important to note that viruses allegedly emerge as prevalent primary opportunistic infections post-transplantation, prompting a re-evaluation of their origins and querying of their endogenous, exogenous, or dual classification.²¹ It is essential to discern the categories of viruses under investigation within this discussion. The contemporary discourse surrounding the purported involvement of the Epstein-Barr virus in multiple sclerosis reveals a notable deviation from scientific rigor. Instead of isolating the virus, researchers have identified the antibodies linked to it.²² This notion is challenged based on recent research, which has previously demonstrated the detection of antibodies against endogenous viruses.²³

The source of many of these misrepresentations of viral origins comes from the dominant viewpoint, that Koch's postulates are applicable in discovering the viral agent behind a disease.²⁴ This is rejected because viruses interact with the host genome, which makes ex vivo culture difficult. Intricate host-pathogen interactions give rise to a dynamic spectrum in which the host functions as an incubator for exogenous viral entities to engage with a diverse spectrum of host genetic elements, including EANS, consequently influencing the intricacies of infection dynamics.

Prior investigations, which focused on alterations observed in the principal component analysis of long-term patients diagnosed with RRMS, directed the focus towards notable shifts in eansRNA ecosystems in response to therapeutic interventions, including ISDs. In the present study, the intent was to assess the potential impact of diet on an individual's RNA ecosystem as an initial strategy for preventing chronic diseases.²⁵

In conclusion, the utilization of these models signifies significant progress in the area of medical surveillance, offering an innovative method for identifying deviations in the eansRNA ecosystem and forecasting subsequent outcomes, such as biomarkers linked to ISD monitoring. It is anticipated that this clinical application will empower healthcare providers with additional time to respond effectively to ISD therapy. Through the surveillance of changes in eansRNA ecosystems, the fundamental origins of most diseases are being investigated, rather than merely observing their symptomatic manifestations. Through the application of state-of-the-art technologies and analytical methodologies, these models provide a promising pathway for improving patient care and therapeutic outcomes in the domain of chronic ailments. Integration of ISD therapy monitoring will further enhance the efficacy and applicability of these models in clinical practice.

### E. References

1. Oellerich, Michael, Karen Sherwood, Paul Keown, Ekkehard Schutz, Julia Beck, Johannes Stegbauer, Lars Christian Rump, and Philip D. Walson. “Liquid biopsies: donor-derived cell-free DNA for the detection of kidney allograft injury.” Nature Reviews Nephrology 17, no. 9 (2021): 591-603.
2. Bhatti G K, Khullar N, Sidhu I S, et al. Emerging role of non-coding RNA in health and disease. Metab Brain Dis. 2021; 36(6):1119-1134. doi:10.1007/s11011-021-00739-y
3. Urnovitz, Howard B., and William H. Murphy. “Human endogenous retroviruses: nature, occurrence, and clinical implications in human disease.” Clinical microbiology reviews9.1 (1996): 72-99.
4. Johnson M, Zaretskaya I, Raytselis Y, Merezhuk Y, McGinnis S, Madden T L. NCBI BLAST: a better web interface. Nucleic Acids Res. 2008; 36(Web Server issue):W5-W9. doi:10.1093/nar/gkn201
5. Chen, Dong-Sheng, et al. “Horizontal gene transfer events reshape the global landscape of arm race between viruses and Homo sapiens.” Scientific reports 6.1 (2016) 26934.
6. Kaur J, Salehen N, Norazit A, et al. Tumor-suppressive effects of GAS5 in cancer cells. Noncoding RNA. 2022; 8(3):39. Published 2022 May 28. doi:10.3390/ncrna8030039
7. Sayad A, Najafi S, Hussen B M et al. The Emerging Roles of the β-Secretase BACE1 and the Long Non-coding RNA BACE1-AS in Human Diseases: A Focus on Neurodegenerative Diseases and Cancer. Front Aging Neurosci. 2022; 14:853180. Published 2022 Mar. 21. doi:10.3389/fnagi.2022.853180
8. ACTRIMS Forum 2023—Poster Presentation #PO15. Multiple Sclerosis Journal. 2023; 29(2_suppl):18-242. doi:10.1177/13524585231169437
9. Cardamone G, Paraboschi E M, Solda G, et al. Not only cancer: the long non-coding RNA MALAT1 affects the repertoire of alternatively spliced transcripts and circular RNAs in multiple sclerosis. Hum Mol Genet. 2019; 28(9):1414-1428. doi:10.1093/hmg/ddy438
10. Nociti V, Santoro M. What do we know about the role of lncRNAs in multiple sclerosis?. Neural Regen Res. 2021; 16(9):1715-1722. doi:10.4103/1673-5374.306061
11. Taft, Ryan J., et al. “Non-coding RNAs: regulators of disease.” The Journal of Pathology: A Journal of the Pathological Society of Great Britain and Ireland 220.2 (2010): 126-139.
12. Boada M, López OL, Olazarin J, et al. A randomized, controlled clinical trial of plasma exchange with albumin replacement for Alzheimer's disease: primary results of the AMBAR Study. Alzheimers Dement. 2020 October; 16(10):1412-1425. doi: 10.1002/alz.12137. Epub 2020 Jul. 27. PMID: 32715623; PMCID: PMC7984263.
13. Dobin A, Davis C A, Schlesinger F, Drenkow J, Zaleski C, Jha S, Batut P, Chaisson M, Gingeras T R. STAR: ultrafast universal RNA-seq aligner. Bioinformatics. 2013 Jan. 1; 29(1):15-21. doi: 10.1093/bioinformatics/bts635. Epub 2012 Oct. 25. PMID: 23104886; PMCID: PMC3530905.
14. Patro, R., Duggal, G., Love, M. et al. Salmon provides fast and bias-aware quantification of transcript expression. Nat Methods 14, 417-419 (2017). https://doi.org/10.1038/nmeth.4197
15. MaxWellClinic, PLC. Impact of Therapeutic Plasma Exchange on RNA Biomark Expression Levels in Alzheimer's Patients. ClinicalTrials.gov identifier: NCT06079827. Updated Oct. 12, 2023. Access Jul. 2, 2024. https://clinicaltrials.gov/study/NCT06079827?locStr-Brentwood,%20TN&country=United % 20States&state=Tennessee&city=Brentwood&cond=Alzheimer %20Disease&rank=1
16. Aswad, Amr, et al. “Evolutionary history of endogenous human herpesvirus 6 reflects human migration out of Africa.” Molecular Biology and Evolution 38.1 (2021): 96-107.
17. Prüfer, K., Racimo, F., Patterson, N. et al. The complete genome sequence of a Neanderthal from the Altai Mountains. Nature 505, 43-49 (2014).
18. Moustafa A, Xie C, Kirkness E, et al. The blood DNA virome in 8,000 humans. PLoS Pathog. 2017; 13(3):e1006292. Published 2017 Mar. 22. doi:10.1371/journal.ppat.1006292
19. Sangiovanni, Mara, Ilaria Granata, Amarinder Singh Thind, and Mario Rosario Guarracino. “From trash to treasure: detecting unexpected contamination in unmapped NGS data.” BMC bioinformatics 20 (2019): 1-12.
20. Laura M. Zahn, Filling the gaps. Science 376, 42-43(2022).DOI:10.1126/science.abp8653
21. Cukuranovic J, Ugrenovic S, Jovanovic I, Visnjic M, Stefanovic V. Viral infection in renal transplant recipients. ScientificWorldJournal. 2012; 2012:820621. doi:10.1100/2012/820621
22. Bjornevik, Kjetil, Marianna Cortese, Brian C. Healy, Jens Kuhle, Michael J. Mina, Yumei Leng, Stephen J. Elledge et al. “Longitudinal analysis reveals high prevalence of Epstein-Barr virus associated with multiple sclerosis.” Science 375, no. 6578 (2022): 296-301.
23. Stevens, Roy W., Aldona L. Baltch, Raymond P. Smith, Bruce J. McCreedy, Phyllis B. Michelsen, Lawrence H. Bopp, and Howard B. Urnovitz. “Antibody to human endogenous retrovirus peptide in urine of human immunodeficiency virus type 1-positive patients.” Clinical Diagnostic Laboratory Immunology 6, no. 6 (1999): 783-786.
24. Rivers, Thomas M. “Viruses and Koch's postulates.” Journal of bacteriology 33, no. 1 (1937): 1-12.
25. Zyla-Jackson K, Walton D A, Plafker K S, et al. Dietary protection against the visual and motor deficits induced by experimental autoimmune encephalomyelitis. Front Neurol. 2023; 14:1113954. Published 2023 Mar. 2. doi:10.3389/fneur.2023.1113954.

### Example 3: EANS Test Report

This Example provides an exemplary analysis of a patient sample with an unknown disease.

| | |
|---|---|
| Patient ID | F363 |
| Date | Sep. 30, 2024 |
| Client ID | MaxWell Clinic |
| Clinical Diagnosis | Post-SARS-CoV-2 |

### 1. Summary

Clustering can reveal genetic patterns linked to specific diseases. Patient F363's genomic profile clustered closely with Parkinson's disease at the 95% confidence limit. However, the heat maps were only partial positive, suggesting the patient may represent an early-stage of parkinsonian disease.

No clustering with cancer samples was observed.

| | Apparently | Neurologic | |
|---|---|---|---|
| Analysis | Healthy | Diseases | Cancer |
| Cluster | Negative | Parkinson | Negative |
| Heat map | Negative | Parkinson | NA |
| | | Partial Positive | |

Recommendation: A second blood sample should be collected and analyzed to determine whether there has been any change in this patient's EANS profile.

### 2. Results

a. Cluster Analysis

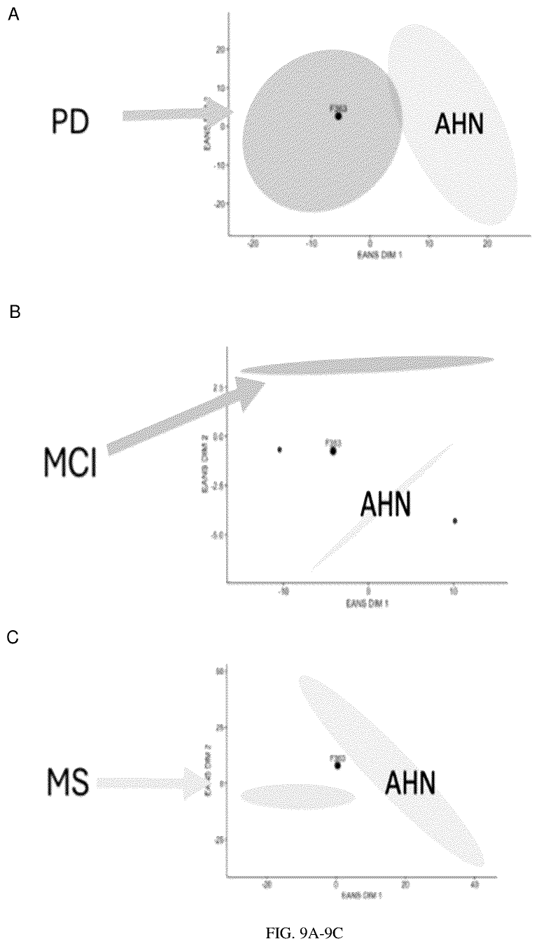

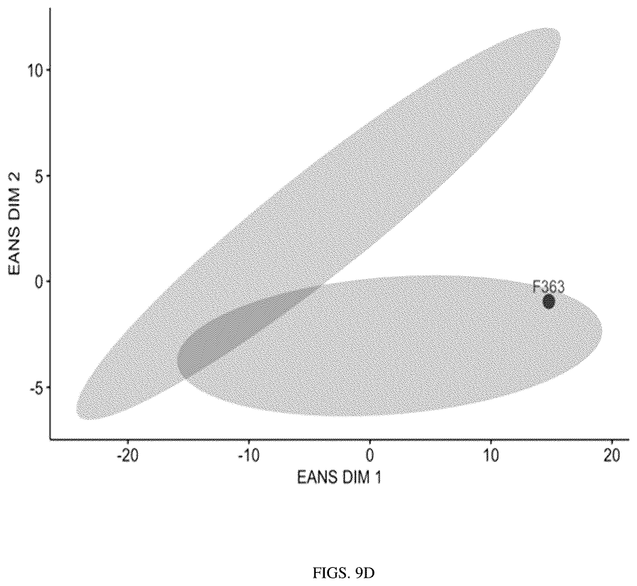

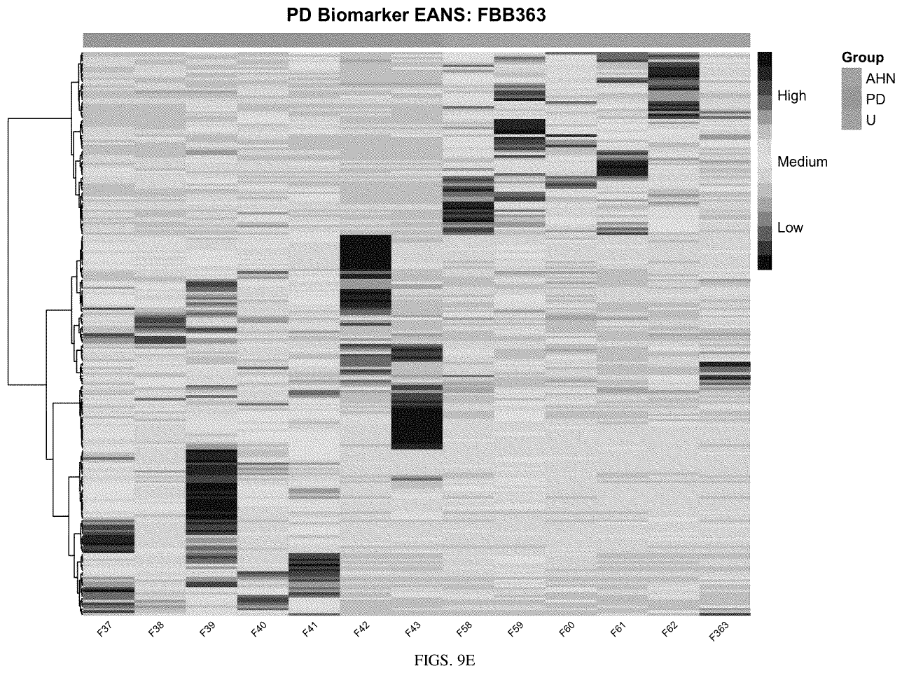

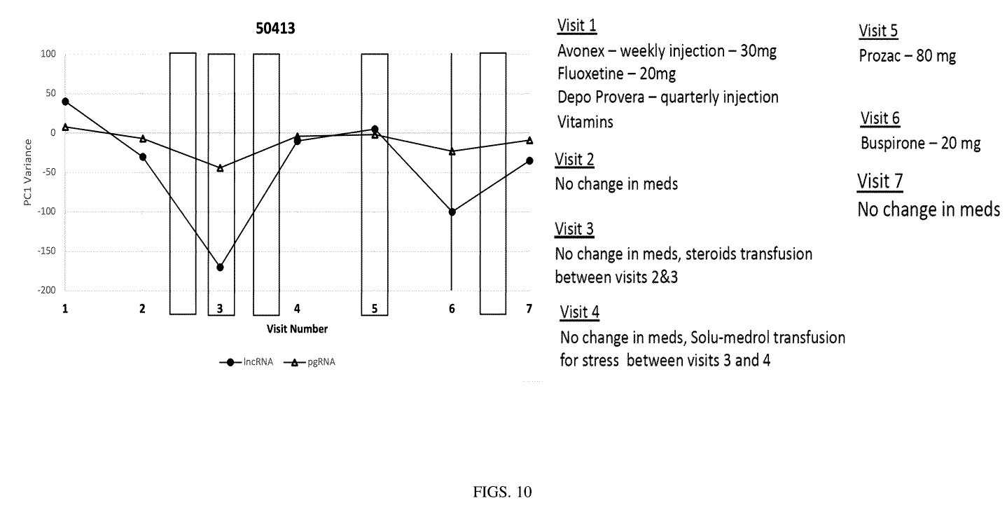

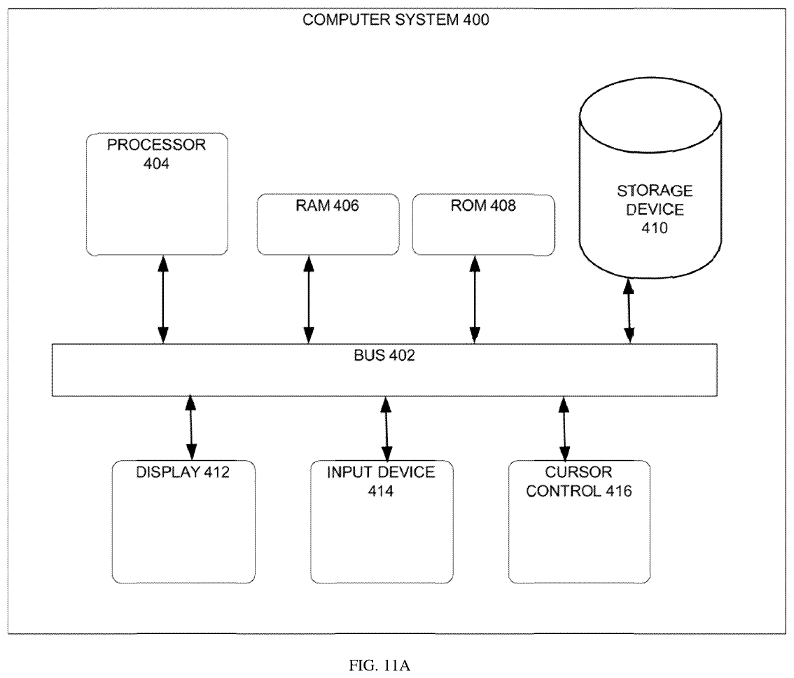

The results in FIGS. 9A-9C are presented as types of membership plots. The patient data are queried against a database of apparently healthy normal controls and individual disease categories databases.

### 1. Neurologic Diseases

Patient F363 data clustered with the Parkinson's database, and not with the apparently healthy normal, MCI, or relapsing-remitting multiple sclerosis databases at 95% confidence. This patient is post SARS-CoV-2 infection and showing grouping with EANS related to PD.

ND Clustering=Parkinsonian

### 2. Cancers

As shown in FIG. 9D, Patient F363 data clustered with the apparently healthy controls database and not with any of the cancers database at 95% confidence. Using the present database, this patient does not appear to have cancer at the time of the blood draw.

Cancer Clustering Negative

b. Heat Map Analysis

The second method of querying the data is to present the results as a heat map of the patient's sample against a selected disease database. In FIG. 9E, data from seven PD patients and five AHN controls were grouped by expression profiles next to the patient's data.

Patient F363 shows partial EANS expression (FIG. 9E). The red arrows indicate statistically unique EANS sequences of patient F363 and 7 different Parkinson's patients. This patient is considered to have a partial positive heat map when clustered with PD patient's data.

PD Heatmap=Partial Positive

### 3. Glossary of Terms

| Term | Description |
|---|---|
| EANS | Endogenous Ancestral Nucleotide Sequence or EANS are panbiotic non- |
| | coding nucleotide sequences that exhibit homology with both human and non- |
| | human genomic sequences, as identified through the Basic Local Alignment |
| | Search Tool (BLAST), a bioinformatics web application for comparing a |
| | query sequence against a database of sequences to find regions of similarity. |
| Membership | A messenger plot in machine learning visually tracks how input features |
| plot | influence outcomes in a model. It highlights relationships and the strength of |
| | influence between variables, helping identify key predictors and interactions. |
| | This tool is especially useful for interpreting complex datasets and |
| | understanding model behavior. |
| PD | “Parkinson's Disease is a progressive disorder that affects the nervous system |
| | and the parts of the body controlled by the nerves. Symptoms start slowly. The |
| | first symptom may be a barely noticeable tremor in just one hand. Tremors are |
| | common, but the disorder also may cause stiffness or slowing of movement.” |
| | (source mayoclinic.org) |
| ND | Neurologic Diseases are disorders that affect the brain as well as the nerves |
| | found throughout the human body and the spinal cord. |
| RUO | Research Use Only test is a label to declare that a company's products can not |
| | be used in diagnostic procedures. |
| RNAseq | RNAseq (RNA-sequencing) is a technique that can examine the quantity and |
| | sequences of RNA in a sample using next-generation sequencing (NGS). Are |
| | you gone |
| MCI | Mild cognitive impairment is the stage between the expected decline in |
| | memory and thinking that happens with age and the more serious decline of |
| | dementia and as is considered an early stage of Alzheimer's disease symptoms. |
| MS | Multiple sclerosis is a disabling disease of the brain and spinal cord (central |
| | nervous system). |
| AHN | The apparently healthy normal term is used as an acknowledgment that some |
| | of the healthy normals may have early-stage asymptomatic diseases. |
| PCA | Principal Component Analysis is a machine learning method used to simplify |
| | a large data set into a smaller set while still maintaining significant patterns |
| | and trends. |
| Analysis | Detailed examination of a patient's RNAseq data using either Cluster, Heat |
| | Map, or Genomic Hotspot. |
| Cluster | A grouping of samples of the same type or disease following ML data |
| | transformation. |
| Heat Map | A data visualization that shows normalized expression values for a large |
| | number of genes simultaneously |
| Genomic | A locus of high activity and variability in the genome that interacts with and |
| Hotspot | promotes or suppresses disease. |
| PAXgene | A specialized blood tube that preserves the RNA from being degraded. |
| tube | |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method comprising:
determining a biological state classification of a subject by inputting sequence data obtained from a sample from a subject to one or more machine learning classifiers, wherein the one or more machine learning classifiers is trained to output biological state classifications based on sequence data of a training data set;
wherein the sequence data comprises the sequences of the RNA isolated from a sample that has been depleted of linear RNA or enriched for double stranded RNA.

2. The method of claim 1, wherein the method further comprises filtering sequence data obtained from a sample from a subject based on long non-coding RNA (lncRNA) and/or pseudogene RNA (pgRNA) and/or a reference genome.

3. (canceled)

4. The method of claim 1, wherein the method further comprises training the machine learning classifier using the training data set, wherein the training data set comprises a filtered sequence profile for each of a plurality of subjects having a known biological state classification, wherein the known biological state classification is one of having a first biological state or not having the first biological state.

5. The method of claim 1, wherein the machine learning classifier comprises a machine learning classifier trained with the training data set, wherein the training data set comprises a filtered sequence profile for each of a plurality of subjects having a known biological state classification, wherein the known biological state classification is one of having a first biological state or having a second biological state.

6. The method of claim 1, wherein determining a biological state classification comprises: generating a report that identifies that the sample evidences the biological state classification.

7-9. (canceled)

10. The method of claim 1, wherein the sequence data comprises a GC content of greater than 55% and/or less than 14% exonic RNA.

11. (canceled)

12. The method of claim 1, wherein the sequence data comprises the nucleotide sequences of RNA fragments that are 35-500 nucleotides in length.

13. The method of claim 1, wherein the sequence data is from RNA extracted from about 1.5-4 mL of blood and/or from 2-5 μg of sequenced RNA.

14. (canceled)

15. The method of claim 1, wherein the sequence data excludes sequences from 3′ polyadenylated RNA and/or sequence from mechanical size-selected RNA.

16-17. (canceled)

18. The method of claim 1, wherein the sample has been depleted of linear RNA by incubation of the RNA isolated from the sample with an exoribonuclease that preferentially hydrolyzes single-stranded RNA.

19. (canceled)

20. The method of claim 18, wherein the exoribonuclease comprises RNAse R.

21-22. (canceled)

23. The method of claim 2, wherein the reference genome comprises a species-specific reference genome and wherein the species of the assembly is the same as the species of the subject and wherein the species comprises H. sapiens.

24. (canceled)

25. The method of claim 1, wherein the subject is a human subject.

26-27. (canceled)

28. The method of claim 1, wherein the machine learning classifier comprises a supervised model that has undergone tuning.

29. The method of claim 1, wherein training the machine learning classifier comprises:
reducing a dimensionality of the machine learning classifier based on a covariance of two or more parameters of the filtered sequence data of the training data set.

30. The method of claim 29, wherein the two or more parameters of the filtered sequence data of the training data set are associated with two or more regions of interest (ROIs) of the filtered sequence data of the training data set.

31. The method of claim 29, wherein the parameters and/or ROIs are non-coding nucleic acid sequences.

32. (canceled)

33. The method of claim 1, wherein the sample comprises urine, fecal, blood, tears, cerebral spinal fluid, feces, or saliva sample.

34. (canceled)

35. A method for treating a subject for a disease, the method comprising treating a subject for the disease, wherein the subject has been determined to have the disease by a trained machine learning classifier that is trained to output biological state classifications based on sequence data of a training data set; wherein the sequence data comprises the sequences of the RNA isolated from a sample that has been depleted of linear RNA or enriched for double stranded RNA.

36-61. (canceled)

62. A method comprising:
i) depleting linear RNA in a biological sample from a subject; and
ii) sequencing the RNA that has been depleted of linear RNA.

63-177. (canceled)
